(19) United States
(12) Patent Application Publication
(10) Pub. No.: US 2022/0088102 A1
(43) Pub. Date: Mar. 24, 2022

(54) CROTON LECHLERI COMPOSITIONS AND THEIR USE IN THE TREATMENT OF CYSTIC FIBROSIS

(71) Applicant: ALPHYN BIOLOGICS, LLC, Cincinnati, OH (US)

(72) Inventors: Gary Michael PEKOE, Cincinnati, OH (US); Jazmyne Kristyne MINK, Cincinnati, OH (US); Steven Aaron PENTELNIK, Cincinnati, OH (US); Neal G. KOLLER, Annapolis, MD (US)

(21) Appl. No.: 17/482,395

(22) Filed: Sep. 22, 2021

Related U.S. Application Data
(60) Provisional application No. 63/081,645, filed on Sep. 22, 2020.
(60) Provisional application No. 63/081,665, filed on Sep. 22, 2020.

(51) Int. Cl. A61K 36/47; A61K 9/00; A61P 31/04

(52) U.S. Cl. CPC A61K 36/47; A61K 2236/53; A61P 31/04; A61K 9/0073

(57) ABSTRACT

The present disclosure provides for the treatment cystic fibrosis and/or a bacterial infection in a cystic fibrosis patient via the administration to the lungs via inhalation of a pharmaceutical composition comprising a therapeutically effective amount of an extract of the Croton lechleri tree. Also provided are details of studies on the effectiveness of an extract of the Croton lechleri tree on cystic fibrosis and/or a bacterial infection in a cystic fibrosis patient and causative pathogens.

### CROSS-REFERENCE TO RELATED APPLICATIONS

This application claims the benefit of U.S. Provisional Application No. 63/081,645 filed Sep. 22, 2020 and U.S. Provisional Application No. 63/081,665 filed Sep. 22, 2020. The disclosures of each of these applications are incorporated herein by reference.

### SUMMARY

The present invention is generally related to the treatment of cystic fibrosis and/or a bacterial infection in a cystic fibrosis patient via the delivery to the lungs a pharmaceutical compositions comprising a therapeutically effective amount via inhalation of latex of Croton lechleri, preferably filtered latex of Croton lechleri, preferably filtered latex of Croton lechleri Müll.Arg., where the components that have a negative impact on the delivery and/or treatment via inhalation treatment have optionally been removed and wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard. The concentration of components and performance standards of latex of Croton lechleri, preferably the concentration of components and performance standards of filtered latex of Croton lechleri, preferably the concentration of components and performance standards of filtered latex of Croton lechleri Müll.Arg of the reference standard are found in Tables 1a-d.

### BRIEF DESCRIPTION OF THE DRAWINGS

FIG. 1 depicts a representative Total Ion Chromatogram as well as additional Multiple Reaction Monitoring spectra that identify the marker compounds in an AB-101 composition.

FIG. 2A depicts the NMR spectra of 3 lots of AB-101 in D₂O—the top spectra is for Lot 00, the middle spectra is for Lot 01, and the bottom spectra is for Lot 02.

FIG. 2B depicts the overlay of the NMR spectra of Lots 00, 01, and 02 of AB-101 in D₂O.

FIG. 3A depicts the Nuclear Magnetic Resonance (NMR) spectra of 3 lots of AB-101 in d₄-Methanol—the top spectra is for Lot 00, the middle spectra is for Lot 01, and the bottom spectra is for Lot 02.

FIG. 3B depicts the overlay of the NMR spectra of Lots 00, 01, and 02 of AB-101 in d₄-Methanol.

FIG. 4A depicts the NMR spectra of 4 lots of AB-101 in d₄-Methanol—the top spectra is for Lot 00, the upper middle spectra is for Lot 01, the lower middle is for Lot 02, and the bottom spectra is for Lot X.

FIG. 4B depicts the overlay of the NMR spectra of Lots 00, 01, 02, and X of AB-101 in d₄-Methanol.

FIG. 5 depicts bar graphs comparing the AB-101 lot analysis results for A) gallocatechin B) epigallocatechin C) catechin D) epicatechin and E) taspine.

FIG. 6 depicts a representative Total Ion Chromatogram of dimethylcedrusin.

FIG. 7 depicts the zone of inhibition of methanol extracted AB-101 against methicillin-susceptible Staphylococcus aureus (MSSA) (on the left) and methicillin-resistant Staphylococcus aureus (MRSA) (on the right).

FIG. 8 depicts the MSSA recovered over time in time-kill kinetic assay.

FIG. 9 depicts the MRSA recovered over time in time-kill kinetic assay.

FIG. 10 depicts the gel permeation chromatogram of each of the 3 PMMA standards.

FIG. 11 depicts the overlay of the gel permeation chromatogram of the 3 PMMA standards.

FIG. 12 depicts the AB-101 Lot 01 chromatograms at a 1.25 mg/mL concentration.

FIG. 13A depicts the calibration curve for M_{w}.

FIG. 13B depicts the calibration curve for Mₙ.

### DEFINITIONS

Before the present compositions and methods are described, it is to be understood that this invention is not limited to the particular processes, compositions, or methodologies described, as these may vary. It is also to be understood that the terminology used in the description is for the purpose of describing the particular versions or embodiments only and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of embodiments herein, the preferred methods, devices, and materials are now described. All publications mentioned herein are incorporated by reference in their entirety. Nothing herein is to be construed as an admission that embodiments herein are not entitled to antedate such disclosure by virtue of prior invention.

As used herein, the terms below have the meanings indicated.

It must also be noted that as used herein and in the appended claims, the singular forms “a,” “an,” and “the” include plural reference unless the context clearly dictates otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean plus or minus 10% of the numerical value of the number with which it is being used. Therefore, about 50% means in the range of 45%-55%.

As used herein, the term “AB-101” maybe used interchangeably with latex of Croton lechleri, preferably filtered latex of Croton lechleri, preferably filtered latex of Croton lechleri Müll.Arg. where the components that have a negative impact on the delivery and/or treatment via inhalation have optionally been removed and botanical raw material. The latex is excreted material from the wounded trunk of Croton lechleri, preferably of Croton lechleri Müll.Arg.

“Administering” when used in conjunction with a therapeutic, such as AB-101, means to administer a therapeutic directly into or onto a target tissue or to administer a therapeutic to a patient whereby the therapeutic positively impacts the tissue to which it is targeted. Thus, as used herein, the term “administering”, when used in conjunction with a composition of embodiments herein, can include, but is not limited to, providing the composition into or onto the target tissue; providing the composition to a patient by, e.g., oral inhalation and/or nasal inhalation whereby the therapeutic reaches the target tissue. “Administering” a composition may be accomplished viai oral inhalation and/or nasal inhalation or in combination with other known techniques.

The transitional term “comprising,” which is synonymous with “including,” “containing,” or “characterized by,” is inclusive or open-ended and does not exclude additional, unrecited elements or method steps.

In embodiments or claims where the term “comprising” is used as the transition phrase, such embodiments can also be envisioned with replacement of the term “comprising” with the terms “consisting of” or “consisting essentially of.”

As used herein, the term “consists of” or “consisting of” means that the pharmaceutical composition, composition or the method includes only the elements, steps, or ingredients specifically recited in the particular claimed embodiment or claim.

As used herein, the term “consisting essentially of” or “consists essentially of” means that the pharmaceutical composition, or the method includes only the elements, steps or ingredients specifically recited in the particular claimed embodiment or claim and may optionally include additional elements, steps or ingredients that do not materially affect the basic and novel characteristics of the particular embodiment or claim. For example, the only active ingredient(s) in the composition or method that treats the specified condition (e.g., nutrient depletion) is the specifically recited therapeutic(s) in the particular embodiment or claim.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “excipient” and “pharmaceutically acceptable excipient” as used herein are intended to be generally synonymous, and is used interchangeably with, the terms “carrier,” “pharmaceutically acceptable carrier,” “diluent,” “pharmaceutically acceptable diluent.”

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

As used herein, the term “pharmaceutically acceptable salt” refers to a salt prepared from a base or acid which is acceptable for administration to a patient, such as a mammal. The term “pharmaceutically acceptable salts” embraces salts commonly used to form alkali metal salts and to form addition salts of free acids or free bases. The nature of the salt is not critical, provided that it is pharmaceutically-acceptable. Such salts can be derived from pharmaceutically-acceptable inorganic or organic bases and from pharmaceutically-acceptable inorganic or organic acids.

As used in each of the embodiments here in, sap may be include among others sap, latex, resin, extract, or any combination of the foregoing.

As used herein, the term “therapeutic” or “therapeutic agent” or “pharmaceutically active agent” means an agent utilized to treat, combat, ameliorate, prevent or improve an unwanted condition or disease of a patient. In part, embodiments of the present invention are directed to the treatment of cystic fibrosis and/or a bacterial infection in a cystic fibrosis patient, including, but not limited to a Pseudomonas aeruginosa infection in a cystic fibrosis patient, a Multi drug resistant (MDR) Pseudomonas aeruginosa infection in a cystic fibrosis patient, a Staphylococcus aureus infection in a cystic fibrosis patient, or a methicillin-resistant Staphylococcus aureus infection in a cystic fibrosis patient.

The term “therapeutically acceptable” refers to those compositions which are suitable for use in contact with the tissues of patients without undue toxicity, irritation, and allergic response, are commensurate with a reasonable benefit/risk ratio, and are effective for their intended use.

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid.

The phrase “therapeutically effective” is intended to qualify the amount of active ingredients used in the treatment of a disease or disorder or on the effecting of a clinical endpoint.

A “therapeutically effective amount” or “effective amount” of a composition is a predetermined amount calculated to achieve the desired effect, i.e., to inhibit, block, or reverse the activation, migration, or proliferation of cells. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. The compounds are effective over a wide dosage range and, for example, dosages per application will normally fall within the range of from 0.001 to 10 mg/kg, more usually in the range of from 0.01 to 1 mg/kg. However, it will be understood that the effective amount administered will be determined by the physician in the light of the relevant circumstances including the condition to be treated, the choice of compound to be administered, and the chosen route of administration, and therefore the above dosage ranges are not intended to limit the scope of the invention in any way. A therapeutically effective amount of the composition of this invention is typically an amount such that when it is administered in a physiologically tolerable excipient composition, it is sufficient to achieve an effective systemic concentration or local concentration in the tissue.

The terms “treat,” “treated,” “treating”, or “treatment” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

Also provided are embodiments wherein any embodiment herein may be combined with any one or more of the other embodiments, unless otherwise stated and provided the combination is not mutually exclusive.

### SUMMARY

The chemical defenses of plants include complex mixtures of organic compounds and typically do not involve individual substances; these compounds appear in different concentrations (majority or minority) within various products derived from natural species. The biological activities of these products can be found to originate from their ability to interact among themselves and other substances through synergistic, additive, antagonistic effects—and can be optimized through the modification of the pharmacokinetics and/or pharmacodynamics of the component substances. The biological effects may occur from the interaction with all the organic compounds or by the interaction among certain components, which may present themselves as majority or minority. Accordingly, when described herein, AB-101 consists of the latex obtained from the Croton lechleri tree but is selected based upon the presence of select components that meet the reference standard as described herein.

In natural product research, the major compound's role and mechanism in its associated biological activity is commonly investigated. Thus, in the scientific literature, there are innumerous published studies where the major components have been found to be responsible for these activities. However, this disregards the possible interactions among the totality of compounds that may be present at lower concentrations in natural products. A study carried out with the essential oil Thymus vulgaris and its major constituent, thymol identified that the effect of the constituents of the oil was not phytotoxic to lettuce seeds, whereas the isolated action of thymol caused significant inhibitory effects on seed germination, raising the possibility of a partial inhibition of thymol activity by other components of the oil. Demonstrating the importance of considering the interactions among all components of the product.

For example, evaluation of the activity of the sap of Dracaena cochinchinensis and three active constituents regarding the analgesic activity from the inhibition of currents on the TRPV1 channel, induced by capsaicin. As a result, the authors found that the combination of the three active components of the sap is responsible for the analgesic activity of the species in question, where these components act synergistically, as the compounds found in greater concentration were not directly responsible for the biological activity found.

Another consideration regarding interactions among the active components of a natural product is the ability to alter the pharmacokinetics of the components when compared with the administration of these molecules in isolation. This can be achieved by modifying the absorption, distribution, metabolism and elimination profiles. A study reported the pharmacokinetic profile of chlorogenic acid and coryloin alone in comparison with the product formed by the hydroalcoholic extract of Pharbitis nil and Corydalis tuber, DA-9701, which contains the two components in equivalent concentrations. Results showed a significant increase in the AUC of coryloin when DA-9701 was administered compared with the two compounds in isolation, both orally. This increase in AUC can be explained by decreased hepatic and/or gastrointestinal first-pass metabolism compared with pure coryloin. In addition, there may be inhibition of corticosteroid presystemic metabolism by other components of DA-9701.

Another example is the complexity of metabolic pathways and the complexity of essential oils, extracts and herbal products may be directly related to the recorded biological effect. In a study with essential oil of Eucalyptus tereticornis and its major constituents, it was observed that all three major constituents reinforce the constricting effect of acetylcholine in the trachea of rats, however with a stimulus of potassium, the essential oil presents a relaxing effect, may be due to the inhibition of acetylcholinesterase activity.

Croton lechleri (a member of the family Euphorbiaceae, commonly called the spurge family) has approximately 1,300 species of plants that are either herbaceous (plants that have no persistent woody stem above ground), shrub (a woody plant which is smaller than a tree and has several main stems arising at or near the ground), tree (a perennial plant with an elongated stem, or trunk, supporting branches and leaves in most species), or liana (any of various long-stemmed, woody vines that are rooted in the soil at ground level and use trees, as well as other means of vertical support, to climb up to the canopy to get access to well-lit areas of the forest) forms. The Croton genus is a diverse and complex group of flowering plants ranging from herbs and shrubs to trees. The Croton genus is widely distributed in tropical and subtropical regions around the world.

Dragon's blood refers to a bright red resin that is obtained from different species of a number of distinct plant genus: Croton, Dracaena, Daemonorops, Calamus rotang and Pterocarpus. The red resin has been in continuous use since ancient times as varnish, medicine, incense, and dye. The name dragon's blood is used to refer to all of the above plant genus, often without any distinction as to the genus or species it is coming from. Those with the same genus will be similar in any therapeutic or nutritional value, with factors such as local soil, local rainfall, local humidity, local sunlight, local fauna and the like imparting variability and inconsistency. However, the difference between the red resin coming from Croton versus Daemonorops (a genus of rattan palms in the family Arecaceae found primarily in the tropics and subtropics of southeastern Asia with a few species extending into southern China and the Himalayas) will be significant. The Croton and Daemonorops genus originate from opposite sides of the world so their components are different and therefore specificity of source plant is important to deliver the desired medicinal benefits or avoid undesirable toxic results. For example milky white latex that is often toxic or at least irritating to the skin is common to the members of the spurge or Euphorbiaceae family. Therefore selecting the specific genus, species, and local geographical area of the spurge or Euphorbiaceae family is essential to having the possibilty for the latex to have specific and repetitive medicinal properties.

A handful of Croton species found in the South America rainforest (in countries of Bolivia, Brazil, Colombia, Ecuador and Peru) Central America and Mexico produce the red latex, commonly known as dragon's blood, that has medicinal properties. The dragon's blood trees grown in these areas include Croton lechleri, Croton draco, Croton palanostigma, Croton sordidus, Croton urucurana, and Croton xalapenesis.

In certain embodiments, the specific dragon's blood tree of the present application is Croton lechleri Müll.Arg. of the Family: Euphorbiaceae. Dragon's blood is also referred to as Sangre de drago (Peru), Sangre de grado (Ecuador).

While the desired medicinal properties could be found by extracting the compositions from either the leaves or bark, in preferred embodiments, it is the deep red latex of Croton lechleri, preferably filtered latex of Croton lechleri, preferably filtered latex of Croton lechleri Müll.Arg where the components that have a negative impact on the delivery and/or treatment via inhalation have optionally been removed and wherein the composition contains at least the concentration of components of latex of Croton lechleri, preferably the concentration of components of filtered latex of Croton lechleri, preferably the concentration of components of filtered latex of Croton lechleri Müll.Arg of the reference standard, that is also referred to as latex, that is utilized. According to Langenheim (2003) resin “is a lipid-soluble mixture of volatile and non-volatile terpenoid and/or phenolic secondary compounds that are usually secreted in specialized structures located either internally or on the surface of the plant and are of potential significance in ecological interactions”. By contrast, latex, is a mixture of terpenoids, phenolic compounds, acids, carbohydrates, etc. having a protective role (Lewisohn 1991) and produced in special cells called laticifers (Fahn 1979). Chemical characterization of dragon's blood is species specific and has been undertaken by many authors. For example, it is possible to distinguish between dragon's blood from some individual species used in works of art, since it has been sold as a colorant for many centuries (Baumer and Dietemann 2010). Dragon's blood of Croton spp. is usually referred to as latex due to the fact that it is secreted and stored by laticifers, and its major constituents are polymeric anthocyanidins, which co-occur with many minor constituents, including diterpenes and simple phenols (Salatino et al. 2007). Dragon's blood secreted by stems of Pterocarpus officinalis is also called latex (Weaver 1997; Guerrero and Guzman 2004); however, information about the chemical composition of the exudate and its ecological function is poorly known. Dragon's blood derived from species of Dracaena and Daemonorops is a phenolic resin (Langenheim 2003), with well-recognized chemical content (e.g. Gonzalez et al. 2000; Shen et al. 2007; Sousa et al. 2008). Sometimes, dragon's blood is referred to as latex (e.g. Philipson 2001). However, this could prove to be a source of confusion, since plants produce other exudates referred to by that name, such as xylem latex and phloem latex, which are entirely different in terms of their location, chemical composition and function. The resin is obtained through tapping the tree or other common draining methods. Draining the tree latex has the additional benefit of not having to use complex and costly extraction technology to obtain the desired composition from either the leaves or bark. The latex of Croton lechleri Müll.Arg. of the present application is then filtered in a 30 micron filter to remove plant debris and thick, resinous material. Chemical characterization of dragon's blood is local geography specific and has not been undertaken by prior authors.

Medicinal and toxic properties of various species of the Croton genus have been ascribed to a wide variety of chemical compounds, such as terpenoids and steroids, alkaloids, and phenolic compounds, the latter including predominantly flavonoids, lignans, and proanthocyanidins. Some embodiments of the present application utilize the whole latex, thereby leveraging the “organic” synergy of all the latex components as intended by nature. The molecular classes found in latex of Croton lechleri Müll.Arg. of the present application which provide the desired medicinal benefits of Croton lechleri Müll.Arg. are: Alkaloids, Diterpenes, Lignans, Phenols, Phytosterols, Proanthocyanidins, Sterols and Tannins.

AB-101 is a novel first-in-class of a new class of topical antibiotics called Multi-Target Therapeutics (MTT). The AB-101 platform utilizes a unique and very specific, well characterized latex from the Croton lechleri Müll.Arg tree that is native to South America. The latex and therefore AB-101 Botanical Drug Substance (BDS) has multiple bioactive compounds using a unique latex with novel performance efficacy.

AB-101 has unique antibiotic properties as demonstrated via bioassay testing demonstrating AB-101 is effective against the gram-positive pathogens associated with Staphylococcus aureus (S. aureus), methicillin-resistant Staphylococcus aureus (MRSA), Streptococcus pyogenes (S. pyogenes) and the gram-negative pathogen of Pseudomonas aeruginosa (P. aeruginosa). The fact that AB-101 has efficacy against both gram-positive and gram-negative pathogens is unique when compared to the typical specifically synthetically derived active for a drug compound, and this benefit is directly attributed to AB-101 MTT properties.

MTT affords AB-101 a broad, multi-mechanism mode of action, which, in turn, strongly reduces the potential for development of bacterial resistance and provides broad-spectrum activity against many different bacteria. The alarming need for new, effective treatments, combined with the increasing resistance to current standard of care treatment options creates a significant need for an AB-101 topical antibiotic. AB-101's strong antibiotic and safety profile makes it an ideal drug for applications to treat cystic fibrosis.

Mayo Clinic describes Cystic fibrosis (CF) as an inherited disorder that causes severe damage to the lungs, digestive system and other organs in the body. CF damages the airways (bronchiectasis) and causes chronic infections, growths in the nose (nasal polyps), coughing up blood (hemoptysis), respiratory failure, and acute exacerbation are all conditions associated with CF that are inherent and damaging to the bronchial pathway. Cystic fibrosis affects the cells that produce mucus, sweat and digestive juices. These secreted fluids are normally thin and slippery. But in people with CF, a defective gene causes the secretions to become sticky and thick. Instead of acting as lubricants, the secretions plug up tubes, ducts and passageways, especially in the lungs and pancreas. People living with CF are at greater risk of getting lung infections than those who don't have the disease. That's because thick, sticky mucus builds up in their lungs, allowing germs to thrive and multiply, which leads to decrease lung surface area/lung capacity.

Coutinho reports that the most serious symptoms of CF are observed in the lungs, augmenting the risk of bacterial infection. Specifically, B. cepacia, P. aeruginosa and S. aureus are the most important infectious agents in cystic fibrosis patients. During the first decade of life of CF patients, Staphylococcus aureus and Hemophilus influenzae are the most common bacteria isolated from the sputum, but in the second and third decade of life, Pseudomonas aeruginosa is the prevalent bacteria. (Henrique Douglas M Coutinho, Vivyanne S Falcão-Silva and Gregório Fernandes Gonçalves, Pulmonary bacterial pathogens in cystic fibrosis patients and antibiotic therapy: a tool for the health workers, International Archives of Medicine 2008, 1:24).

As highlighted by Coutinho, Staphylococcus aureus is the first pathogen to infect and colonize the airways of CF patients, being the most common pathogen. This microorganism is prevalent in children and may cause epithelial damage, opening the way to the adherence of other pathogens such as Pseudomonas aeruginosa. However, other studies indicate that S. aureus is a co-infective pathogen associated with P. aeruginosa. Together, the inflammatory process is more intense due the additive effect of these two pathogens and the body's response.

Methicillin-resistant S. aureus (MRSA) has become a major nosocomial pathogen with a progressive increase in prevalence also in CF populations. Moreover, the increased frequency of this organism in the community, especially with carriage of virulence factors, is a matter of concern. MRSA is a global problem representing risks to all CF patients.

Valenza further confirms the findings of Coutinho by conducting a study showing analysis of the sputum from CF patients during a period of 12 months indicated the presence of P. aeruginosa in 50% of these individuals, S. aureus in 63.3%, (Valenza G, Tappe D, Turnwald D, Frosch M, König C, Hebestreit H, Abele-Horn M: Prevalence and antimicrobial susceptibility of microorganisms isolated from sputa of patients with cystic fibrosis. J Cyst Fibros 2008, 7(2):123-127).

The Journal of the American Medical Association (JAMA) reported the prevalence of methicillin-resistant Staphylococcus aureus (MRSA) in the respiratory tract of individuals with CF has increased dramatically. They concluded that the detection of MRSA in the respiratory tract of CF patients was associated with worse survival. (Elliott C. Dasenbrook, MD, MHS; William Checkley, MD, PhD; Christian A. Merlo, MD, MHS, Michael W. Konstan, MD; Noah Lechtzin, MD, MHS; Michael P. Boyle, MD, Association Between Respiratory Tract Methicillin-Resistant Staphylococcus aureus and Survival in Cystic Fibrosis, JAMA. 2010; 303(23):2386-2392).

In all the reported studies the presence of Staphylococcus aureus and Pseudomonas aeruginosa are common and prevalent pathogens associated with CF. As presented, the presence of S. aureus is a co-infective pathogen associated with P. aeruginosa. Concomitant with this problem to those with CF is the risk of acquiring MRSA.

Systemic drug medications are some of the primary treatment modes for treating CF infections. Since the two main pathogens are gram-positive and gram-negative, this will typically require multiple drugs. By their very design, CF systemic antibiotic drugs cannot target the pathogens specifically at the site of infections, so the entire body and organs are exposed to these treatments. There are two unfortunate results of the use of systemic antibiotics. First, the systemic drugs put a toxin load on the entire body as well as a limitation on the local dose concentration level of a systemic antibiotic to effectively kill the bacteria. Second, exposing the body and the pathogens to these antibiotics presents the unfortunate risk of initiating multi-drug resistance (MDR), thereby increasing the risk for MRSA and other MDR complications.

AB-101 has unique and novel properties to be an effective treatment for CF. This is the first time these benefits have been discovered to those skilled in the art. AB-101 has shown in bioassays that it is effective against S. aureus and P. aeruginosa. In those cases where MRSA infection is present, AB-101 has shown high efficacy against these pathogens. A novel benefit of AB-101 is that it is effective against both pathogens in one drug. This can enable AB-101 to also act as a prophylactic against P. aeruginosa when treating S. aureus. In the situation when a patient is infected with both pathogens simultaneously, only AB-101 may be required as a single drug.

Since AB-101 uses the entire latex of Croton lechleri Müll.Arg. AB-101 contains significant and critical levels of taspine and dimethylcedrusin (DMC). Taspine and DMC provide epithelial healing benefit for CF patients. As stated by Coutinho, damage to epithelial cells are one of the side effects of bronchial infections of the lungs. The repair of epithelial cells is an important healing characteristic of AB-101. By using the entire latex to leverage the synergies, AB-101 maximizes the MTT performance.

The epithelial cell benefits of taspine and DMC has been shown by Pieters et al (1995) in his paper on the in vivo wound healing activity of the Croton lechleri latex (also called Dragon's Blood). The wound healing activity of the latex, and some of its constituents, including the alkaloid taspine, the dihydrobenzufuran lignan 3′,4-0-dimethylcedrusin and proanthocyanidins (PAC), were evaluated in vivo in rats and compared with the wound healing activity of synthetic proanthocyanidins. The beneficial effect of dragon's blood on wound healing was confirmed. The Croton lechleri latex stimulated contraction of the wound, formation of a crust, formation of new collagen, and regeneration of the epithelial layer. 3′,4-0-dimethylcedrusin also improved wound healing in vivo by stimulating the formation of fibroblasts and collagen, but crude latex was more effective. This was due to the proanthocyanidins and other constituents present in the latex, which stimulate contraction of the wound and precipitate with proteins forming a dark crust covering the wound. However, when tested alone, the synthetic proanthocyanidins showed delay wound repair by a decreased formation of new fibroblasts. (L Pieters 1, T De Bruyne, B Van Poel, R Vingerhoets, J Totté, D Vanden Berghe, A Vlietinck, In vivo wound healing activity of Dragon's Blood (Croton spp.), a traditional South American drug, and its constituents, 1995 Jul; 2(1):17-22).

This demonstrates that the synthetic proanthocyanidins alone delay wound repair, but the involvement of taspine and DMC enhances wound repair, thus overcoming any activity of PACs alone, showing synergy among the different components of the Croton spp. Thus, taspine and DMC are critical components for repair to the lesions in the lung, re-epithelization of the lung tissue, stimulating migration and formation of fibroblasts and other components of lesion repair. AB-101 contains these critical components, separating AB-101 from other forms of Dragon's Blood, Sangre de Grado (SDG) or fractionations of SDG that intend to isolate, fractionate, and purify to isolate primarily the PAC components.

The AB-101 treatment is not intended to be limited to being directly delivered via inhalation to the lungs, but also to be delivered topically to the nose, mouth and throat. The advantage of topical delivery is to provide direct application to the site of infection while having minimal exposure to the entire body. This will decrease the antibiotic load on the entire body while maximizing bacterial treatment to the bronchial system. The benefit of delivering AB-101 beyond the lungs and inclusive to the nose, mouth and throat is to treat and kill any bacteria pathogens that can be spread and colonized in these areas. CF patients have a lot of coughing due to mucus build-up in the lungs, throat and stomach. The result of the sputum as shown by Valenza indicate that the pathogens can be spread throughout the bronchial tract.

The bronchial delivery of AB-101 can be based on using the pure filtered Croton lechleri Müll.Arg latex that meets the unique and very specific characterization targets for the AB-101 in the forms of and not limited to a liquid, lyophilized, rehydrated lyophilized, spray dried, powered, concentrated, reconstituted or other forms familiar to those skilled in the art. AB-101 can be used at full dose level, diluted dose level or concentrated dose levels to those skilled in the art to deliver the desired outcome. AB-101 can be delivered to the bronchial area of the lungs, nose, mouth and throat by and not limited to a nebulized form, aerosolized spray form that is powered either electrostatically, motorized or by compressed gas propellent that aerosolizes the AB-101 or by a mechanically activated form through squeezing or pumping into aerosolize particles appropriate for delivery into the bronchial passageway. In all cases the selection and design of the delivery system is one that maximizes the delivery of AB-101 and ensures easy compliance for the medical professional and for the CF patient.

Some embodiments herein are directed to a method of identifying a composition of latex of Croton lechleri, preferably a composition of filtered latex of Croton lechleri, preferably a composition of filtered latex of Croton lechleri Müll.Arg comprising: (a) determining the concentration of components of latex of Croton lechleri, preferably the concentration of components of filtered latex of Croton lechleri, preferably the concentration of components of filtered latex of Croton lechleri Müll.Arg; (b) comparing the concentrations of the components to the concentrations of the components of a reference standard; and (c) identifying a composition of latex of Croton lechleri, preferably a composition of filtered latex of Croton lechleri, preferably a composition of filtered latex of Croton lechleri Müll.Arg, wherein the composition contains at least the concentration of components of latex of Croton lechleri, preferably the concentration of components of filtered latex of Croton lechleri, preferably the concentration of components of filtered latex of Croton lechleri Müll.Arg of the reference standard.

Some embodiments herein are directed to a method of identifying a composition of latex of Croton lechleri, preferably a composition of filtered latex of Croton lechleri, preferably a composition of filtered latex of Croton lechleri Müll.Arg for use in treating cystic fibrosis and/or a bacterial infection in a cystic fibrosis patient comprising: (a) determining the concentration of components of latex of Croton lechleri, preferably the concentration of components of filtered latex of Croton lechleri, preferably the concentration of components of filtered latex of Croton lechleri Müll.Arg; (b) comparing the concentrations of the components to the concentrations of the components of a reference standard; and (c) identifying a composition of latex of Croton lechleri, preferably a composition of filtered latex of Croton lechleri, preferably a composition of filtered latex of Croton lechleri Müll.Arg where the components that have a negative impact on the delivery and/or treatment via inhalation have optionally been removed for use in treating cystic fibrosis and/or a bacterial infection in a cystic fibrosis patient, wherein the composition contains at least the concentration of components of latex of Croton lechleri, preferably the concentration of components of filtered latex of Croton lechleri, preferably the concentration of components of filtered latex of Croton lechleri Müll.Arg of the reference standard.

Embodiments of the present invention are directed to pharmaceutical compositions of latex of Croton lechleri, preferably filtered latex of Croton lechleri, preferably filtered latex of Croton lechleri Müll.Arg. where the components that have a negative impact on the delivery and/or treatment via inhalation have optionally been removed and a pharmaceutically acceptable excipient. Such pharmaceutical compositions have been found to be useful in the successful treatment of cystic fibrosis and/or a bacterial infection in a cystic fibrosis patient using the same. In some embodiments the pharmaceutical compositions are administered to the lungs. Embodiments are directed to pharmaceutical compositions comprising latex of Croton lechleri, preferably filtered latex of Croton lechleri, preferably filtered latex of Croton lechleri Müll.Arg. where the components that have a negative impact on the delivery and/or treatment via inhalation have optionally been removed disclosed herein together with a pharmaceutically acceptable carrier, as well as methods of making and using the compositions. Certain embodiments are directed to methods for inhibiting cystic fibrosis and/or a bacterial infection in a cystic fibrosis patient. Other embodiments are directed to methods for treating acute bacterial skin or skin structure infections in a patient in need of such treatment, comprising administering to said patient a therapeutically effective amount of a composition according to the present invention. Also provided is the use of certain extracts of Croton lechleri disclosed herein in the manufacture of a medicament for the treatment of cystic fibrosis and/or a bacterial infection in a cystic fibrosis patient.

In certain embodiments, the specific dragon's blood tree of the present application is Croton lechleri Müll.Arg. of the Family: Euphorbiaceae. Dragon's blood is also referred to as Sangre de drago (Peru), Sangre de grado (Ecuador). Embodiments of the present invention are directed to pharmaceutical compositions of latex of Croton lechleri, preferably filtered latex of Croton lechleri, preferably filtered latex of Croton lechleri Müll.Arg where the components that have a negative impact on the delivery and/or treatment via inhalation have optionally been removed and a pharmaceutically acceptable excipient. Such pharmaceutical compositions have been found to be useful in the successful treatment of cystic fibrosis and/or a bacterial infection in a cystic fibrosis patient using the same. In some embodiments the pharmaceutical compositions are administered via oral and/or nasal inhalation. Embodiments are directed to pharmaceutical compositions comprising latex of Croton lechleri, preferably filtered latex of Croton lechleri, preferably filtered latex of Croton lechleri Müll.Arg where the components that have a negative impact on the delivery and/or treatment via inhalation have optionally been removed and wherein the composition contains at least the concentration of components of latex of Croton lechleri, preferably the concentration of components of filtered latex of Croton lechleri, preferably the concentration of components of filtered latex of Croton lechleri Müll.Arg of the reference standard, disclosed herein together with a pharmaceutically acceptable carrier, as well as methods of making and using the compounds and compositions. Other embodiments are directed to methods for treating cystic fibrosis and/or a bacterial infection in a cystic fibrosis patient in need of such treatment, comprising administering to said patient a therapeutically effective amount of a composition according to the present invention. Also provided is the use of certain extracts of Croton lechleri disclosed herein in the manufacture of a medicament for the treatment of cystic fibrosis and/or a bacterial infection in a cystic fibrosis patient.

### Pharmaceutical Compositions

Embodiments herein are directed to pharmaceutical compositions comprising latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the composition contains at least the concentration of components of latex of Croton lechleri, preferably the concentration of components of filtered latex of Croton lechleri, preferably the concentration of components of filtered latex of Croton lechleri Müll.Arg of the reference standard, which have been found to be useful in the successful treatment of cystic fibrosis and/or a bacterial infection in a cystic fibrosis patient.

Importantly, the variation of all the compounds vary greatly as indicated by the geography, environment, soil, elevation and age of the trees to name a few of the key variables. These variations are highlighted by Thiago Vaz Lopes, Dragon's blood (Croton lechleri Müll.Arg; An Update on the Chemical Composition and Medical Applications of This Natural Plant Extract, Revista Brasileira de Higiene and Animal Health (v.7, n2) p. 167-192 (2013)).

The AB-101 novelty is based upon identifying the linkage between the specific compounds and their levels of concentration within AB-101 via a bioassay to in vitro efficacy and confirming via human use testing as an effective treatment for wound treating, bleeding treatment, and fighting infections. The utility of this novel discovery is the basis for developing a pharmaceutical drug and a medicinal product that will meet the FDA standards.

The FDA has established the requirement of having a bioassay that correlates the performance of the botanical raw material based on the chemical characterization of the composition and changes therein, to the efficacy against wound treating, bleeding treatment, and fighting infections.

The Croton lechleri Müll.Arg latex latex is complex, difficult and not straightforward to define since its composition uses the full accompaniment of all of the bioactive materials comprising the Croton lechleri Müll.Arg latex. Net, finding the critical active markers and performance and safety tests requires novel discovery.

The FDA requires the identification of the critical biomarkers or active constituents that drives the bioactivity. To that end, the critical biomarkers and their associated concentrations for AB-101 have never been published, defined or identified as associated with wound healing properties, antimicrobial activity and safety for treatment of wound treating, bleeding treatment, and fighting infections. Without this information, the FDA will not grant a drug status for medicinal use which is at the heart of becoming a pharmaceutical drug.

“Pharmaceutical Products” means any product, compound, medicine or therapeutic which is subject to regulation as a drug, medicine or controlled substance by a foreign equivalent of the United States Food and Drug Administration.

FDA Guidance on Botanicals States:

Because of the heterogeneous nature of a botanical drug and possible uncertainty about its active constituents, one of the critical issues for botanical drugs is ensuring that the therapeutic effect for marketed drug product batches is consistent. In general, therapeutic consistency can be supported by a “totality of the evidence” approach, including the following considerations:
Botanical raw material control (e.g, agricultural practice and collection).
Quality control by chemical test(s) (e.g., analytical tests such as spectroscopic and/or chromatographic methods that capture the active chemical constituents of a botanical drug substance) and manufacturing control (e.g., process validation).
Biological assay (e.g. a biological assay that reflects the drug's known or intended mechanism of action) and clinical data (for details regarding use of clinical data in ensuring therapeutic consistency.

By using the whole Croton lechleri Müll.Arg latex a unique synergy can be obtained across the entire composition that meets the specific bioassay performance targets. Table 1 shows 20 bioactives found in the pharmaceutical grade of AB-101. These bioactive compounds provide efficacy for: antimicrobial, antiviral, anti-inflammatory, cell proliferation to promote healing, anticancer, hemostatic, antioxidant and fibroblast stimulation to promote healing. By maintaining the Croton lechleri Müll.Arg latex intact, a tremendous synergy is obtained across wound healing and preventing infections.

Table A shows bioactive compounds found in the whole Croton lechleri Müll.Arg latex of AB-101 and their properties

TABLE A

| | | | | | Cell | | | | Stimulating |
|---|---|---|---|---|---|---|---|---|---|
| Bioactive | Phytochemical | | | | Proliferative | | | | fibroblasts |
| Chemical | Compound | | | Anti- | (Wound | | | | (Wound |
| Components | Class | Antimicrobial | Antiviral | inflammatory | Healing) | Anticancer | Hemostat | Antioxidant | Healing) |
| 1,3,5- | Flavonoid | X | | | | | | | |
| Trimethyoxy | | | | | | | | | |
| benzene | | | | | | | | | |
| 2,4,6- | Phenol | X | X | | | X | | | |
| Trimethoxyphenol | | | | | | | | | |
| 3′,4-O- | Lignin | | | | | | | | X |
| dimethylcedrusin | | | | | | | | | |
| 4-O- | Lignin | | | | X | | | | |
| Dimethylcedrusin | | | | | | | | | |
| Boldine, iso | Alkaloid | | | X | | X | | | |
| Catechin | Flavonoid | X | X | X | | | X | X | |
| Epicatechin | Flavonoid | | X | X | | | | X | |
| Epigallocatechin | Phenol | X | | | | | | X | |
| Flavan-3-ols | Flavonoids | X | X | | | | | | |
| Gallocatechin | Flavonoid | X | X | | X | | | X | |
| Magnoflorine | Alkaloid | X | X | X | | | | | |
| Proanthocyanidins | Polyphenols | X | X | X | X | X | | X | |
| Procyanidin | Flavonoids | X | X | X | X | | | | |
| Prodelphinidin | Tannins | X | | | | | | | |
| Sitosterol- | Phytosterol | X | X | X | | | | | |
| Beta- | | | | | | | | | |
| Glucopyranoside | | | | | | | | | |
| Taspine | Alkaloid | X | X | X | | X | | | |
| Catechin | Polyphenols | X | X | X | X | X | | X | X |
| gallate | | | | | | | | | |
| Epicatechin | Polyphenols | X | | | X | X | | X | X |
| gallate | | | | | | | | | |
| Epigallocatechin | Polyphenols | X | X | X | X | X | | X | X |
| gallate | | | | | | | | | |
| Gallocatechin | Polyphenols | X | | | X | | | X | X |
| gallate | | | | | | | | | |

Some embodiments herein are directed to a pharmaceutical composition comprising latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the composition contains at least the concentration of components of latex of Croton lechleri, preferably the concentration of components of filtered latex of Croton lechleri, preferably the concentration of components of filtered latex of Croton lechleri Müll.Arg of the reference standard, of embodiments herein and a pharmaceutically acceptable excipient. Optionally, the pharmaceutical composition may further comprise one or more other therapeutic ingredients. In some embodiments, the pharmaceutical composition comprises a therapeutically effective amount of the latex of Croton lechleri, preferably filtered latex of Croton lechleri, preferably filtered latex of Croton lechleri Müll.Arg., where the components that have a negative impact on the delivery and/or treatment via inhalation have optionally been removed and wherein the composition contains at least the concentration of components of latex of Croton lechleri, preferably the concentration of components of filtered latex of Croton lechleri, preferably the concentration of components of filtered latex of Croton lechleri Müll.Arg of the reference standard. In some embodiments, the pharmaceutical composition is suitable for administration to the lungs or is an orally inhalable and/or nasally inhalable pharmaceutical composition. In some embodiments, the pharmaceutical composition is suitable for administration is suitable for administration via inhalation. In some embodiments latex of Croton lechleri, preferably filtered latex of Croton lechleri, preferably filtered latex of Croton lechleri Müll.Arg. has a PDI of embodiments disclosed herein.

Embodiments herein are directed to pharmaceutical compositions comprising latex of Croton lechleri, preferably filtered latex of Croton lechleri, preferably filtered latex of Croton lechleri Müll.Arg., where the components that have a negative impact on the delivery and/or treatment via inhalation have optionally been removed and wherein the pharmaceutical composition does not contain a pharmaceutically acceptable excipient. In certain embodiments, latex of Croton lechleri, preferably filtered latex of Croton lechleri, preferably filtered latex of Croton lechleri Müll.Arg. where the components that have a negative impact on the delivery and/or treatment via inhalation have optionally been removed comprises one or more compounds selected from: gallocatechin, epigallocatechin, catechin, epicatechin, taspine, and dimethylcedrusin and combinations thereof. Each of gallocatechin, epigallocatechin, catechin, epicatechin, taspine, and dimethylcedrusin may be present in the latex of Croton lechleri, preferably filtered latex of Croton lechleri, preferably filtered latex of Croton lechleri Müll.Arg. where the components that have a negative impact on the delivery and/or treatment via inhalation have optionally been removed in at least the amounts found in Table 1a or any combination of such amounts.

Embodiments herein are directed to pharmaceutical compositions comprising latex of Croton lechleri, preferably filtered latex of Croton lechleri, preferably filtered latex of Croton lechleri Müll.Arg. where the components that have a negative impact on the delivery and/or treatment via inhalation have optionally been removed and a pharmaceutically acceptable excipient. In certain embodiments, latex of Croton lechleri, preferably filtered latex of Croton lechleri, preferably filtered latex of Croton lechleri Müll.Arg. where the components that have a negative impact on the delivery and/or treatment via inhalation have optionally been removed comprises one or more compounds selected from: gallocatechin, epigallocatechin, catechin, epicatechin, taspine, and dimethylcedrusin and combinations thereof. Each of gallocatechin, epigallocatechin, catechin, epicatechin, taspine, and dimethylcedrusin may be present in the latex of Croton lechleri, preferably filtered latex of Croton lechleri, preferably filtered latex of Croton lechleri Müll.Arg. where the components that have a negative impact on the delivery and/or treatment via inhalation have optionally been removed in at least the amounts found in Table 1 or any combination of such amounts.

As shown in Table B, there are a broad range of compounds present in AB-101. The primary bioactive reference standard for the pharmaceutical grade of AB-101 are the Gallocatechin, Epigallocatechin, Catechins, Epicatechin, Taspine and Dimethylcedrusin. Of particular importance is a secondary set of polyphenol bioactives composed of the gallate family including Catechin Gallate (CG), Epicatechin Gallate (ECG), Gallocatechin Gallate (GCG) and Epigallocatechin Gallate (EGCG). The gallate family bioactive profile of particular importance to AB-101 include the antimicrobial and antioxidants properties. These properties have been noted and indicated in Rahardiyan, Dino. (2018), Antibacterial potential of catechin of tea (Camellia sinensis) and its applications, Food Research. 3. 1-6, and Multifunctional Antioxidant Activities of Alkyl Gallates The Open Bioactive Compounds Journal, 2010, 3: 1-11 Isao Kubo, Noriyoshi Masuoka, Tae Joung Ha, Kuniyoshi Shimizu, Ken-ichi Nihei.

From a composition standpoint the primary and secondary bioactives compose between 80% to 99% of the concentration composition of the pharmaceutical grade of AB-101, where the remaining other compounds not characterized comprise the remaining whole of AB-101. Within the whole, the gallate bioactive family can contribute between 1% to 20% of the bioactive. For AB-101 Lot 01, the primary bioactive reference range is between 85% to 90%, the secondary reference range is between 3% to 4% and the total compounds not characterized in AB-101 ranges from 7% to 11%.

The contribution of the entire Croton lechleri Müll.Arg latex having a unique synergy across the entire composition that meets the specific bioassay performance targets resulting in a composition that has great natural polydispersity as measured by a Polydispersity Index Analysis. The primary reference standard is the main focus of the pharmaceutical grade of AB-101's bioactivity, where the secondary reference standard demonstrates the biodiversity, the polydispersity and synergy makeup within AB-101, which also contributes to AB-101 efficacy.

TABLE 1a

| | Exemplary Amount present in |
|---|---|
| Compound | the latex (PPM is in μg/g) |
| Gallocatechin | at least about 110 PPM |
| Epigallocatechin | at least about 780 PPM |
| Catechin | at least about 1.6 PPM |
| Epicatechin | at least about 2 PPM |
| Taspine | at least about 45 PPM |
| Dimethylcedrusin | at least about 0.1 PPM |

TABLE 1b

| | Exemplary Amount present in |
|---|---|
| | the latex as a % of total |
| Compound or compounds | Proanthocyanidins (PAC) |
| Gallocatechin and | at least about 60% |
| Epigallocatechin combined | |
| Epigallocatechin | at least about 45% |

TABLE 1c
Exemplary Antibiotic Activity

| Bacteria | Exemplary MIC | Exemplary MBC |
|---|---|---|
| Methicillin-susceptible | 50 μg/mL or less | 50 μg/mL or less |
| Staphylococcus aureus (MSSA) | | |
| Methicillin-resistant | 50 μg/mL or less | 50 μg/mL or less |
| Staphylococcus aureus (MRSA) | | |
| Pseudomonas aeruginosa | 50 μg/mL or less | 50 μg/mL or less |
| Streptococcus pyongenes | 50 μg/mL or less | 50 μg/mL or less |

TABLE 1d
Exemplary LogP for each of gallocatechin, epigallocatechin,
catechin, epicatechin, and taspine

| | |
|---|---|
| ECG LogP: Partition Coefficient Calculation | at least about 2.5 |
| IVPT: Skin Permeation Flux Calculation for ECG | at least about less |
| | than 500 μg/cm²/hr |

If the latex of Croton lechleri, preferably filtered latex of Croton lechleri, preferably filtered latex of Croton lechleri Müll.Arg. where the components that have a negative impact on the delivery and/or treatment via inhalation have optionally been removed fails to contain the amounts of gallocatechin, epigallocatechin, catechin, epicatechin, taspine, and dimethylcedrusin in at least the amounts set forth in Table 1, it is not suitable for use in the pharmaceutical compositions and methods of use described herein.

In some embodiments, the gallocatechin present in the latex is in an amount of at least about 110 PPM, at least about 115 PPM, at least about 120 PPM, at least about 125 PPM, at least about 130 PPM, at least about 135 PPM, at least about 140 PPM, at least about 145 PPM, at least about 150 PPM, at least about 155 PPM, at least about 160 PPM, at least about 165 PPM, at least about 170 PPM, at least about 175 PPM, at least about 180 PPM, at least about 185 PPM, at least about 190 PPM, at least about 195 PPM, at least about 200 PPM, or a range between any two of these values.

In some embodiments, the epigallocatechin present in the latex is in an amount of at least about 780 PPM, at least about 790 PPM, at least about 800 PPM, at least about 810 PPM, at least about 820 PPM, at least about 830 PPM, at least about 840 PPM, at least about 850 PPM, at least about 860 PPM, at least about 870 PPM, at least about 880 PPM, at least about 890 PPM, at least about 900 PPM, at least about 910 PPM, at least about 920 PPM, at least about 930 PPM, at least about 940 PPM, at least about 950 PPM, at least about 960 PPM, at least about 970 PPM, at least about 980 PPM, at least about 990 PPM, at least about 1000 PPM, at least about 1010 PPM, at least about 1020 PPM, at least about 1030 PPM, at least about 1040 PPM, at least about 1050 PPM, at least about 1060 PPM, at least about 1070 PPM, at least about 1080 PPM, at least about 1090 PPM, at least about 1100 PPM, at least about 1110 PPM, at least about 1120 PPM, at least about 1130 PPM, at least about 1140 PPM, at least about 1150 PPM, at least about 1160 PPM, at least about 1170 PPM, at least about 1180 PPM, at least about 1190 PPM, at least about 1200 PPM, at least about 1210 PPM, at least about 1220 PPM, at least about 1230 PPM, at least about 1240 PPM, at least about 1250 PPM, at least about 1260 PPM, at least about 1270 PPM, at least about 1280 PPM, at least about 1290 PPM, at least about 1300 PPM, at least about 1310 PPM, at least about 1320 PPM, at least about 1330 PPM, at least about 1340 PPM, at least about 1350 PPM, at least about 1360 PPM, at least about 1370 PPM, at least about 1380 PPM, at least about 1390 PPM, at least about 1400 PPM, at least about 1410 PPM, at least about 1420 PPM, at least about 1430 PPM, at least about 1440 PPM, at least about 1450 PPM, at least about 1460 PPM, at least about 1470 PPM, at least about 1480 PPM, at least about 1490 PPM, at least about 1500 PPM, at least about 1510 PPM, at least about 1520 PPM, at least about 1530 PPM, at least about 1540 PPM, at least about 1550 PPM, at least about 1560 PPM, at least about 1570 PPM, at least about 1580 PPM, at least about 1590 PPM, at least about 1600 PPM, at least about 1610 PPM, at least about 1620 PPM, at least about 1630 PPM, at least about 1640 PPM, at least about 1650 PPM, at least about 1660 PPM, at least about 1670 PPM, at least about 1680 PPM, at least about 1690 PPM, at least about 1700 PPM, or a range between any two of these values.

In some embodiments, the catechin present in the latex is in an amount of at least about 1.6 PPM, at least about 1.7 PPM, at least about 1.8 PPM, at least about 1.9 PPM, at least about 2.0 PPM, at least about 2.1 PPM, at least about 2.2 PPM, at least about 2.3 PPM, at least about 2.4 PPM, at least about 2.5 PPM, at least about 2.6 PPM, at least about 2.7 PPM, at least about 2.8 PPM, at least about 2.9 PPM, at least about 3.0 PPM, at least about 3.1 PPM, at least about 3.2 PPM, at least about 3.3 PPM, at least about 3.4 PPM, at least about 3.5 PPM, at least about 3.6 PPM, at least about 3.7 PPM, at least about 3.8 PPM, at least about 3.9 PPM, at least about 4.0 PPM, at least about 4.1 PPM, at least about 4.2 PPM, at least about 4.3 PPM, at least about 4.4 PPM, at least about 4.5 PPM, at least about 4.6 PPM, at least about 4.7 PPM, at least about 4.8 PPM, at least about 4.9 PPM, at least about 5.0 PPM, at least about 5.1 PPM, at least about 5.2 PPM, at least about 5.3 PPM, at least about 5.4 PPM, at least about 5.5 PPM, at least about 5.6 PPM, at least about 5.7 PPM, at least about 5.8 PPM, at least about 5.9 PPM, at least about 6.0 PPM, at least about 6.1 PPM, at least about 6.2 PPM, at least about 6.3 PPM, at least about 6.4 PPM, at least about 6.5 PPM, at least about 6.6 PPM, at least about 6.7 PPM, at least about 6.8 PPM, at least about 6.9 PPM, at least about 7.0 PPM, at least about 7.1 PPM, at least about 7.2 PPM, at least about 7.3 PPM, at least about 7.4 PPM, at least about 7.5 PPM, at least about 7.6 PPM, at least about 7.7 PPM, at least about 7.8 PPM, at least about 7.9 PPM, at least about 8.0 PPM, at least about 8.1 PPM, at least about 8.2 PPM, at least about 8.3 PPM, at least about 8.4 PPM, at least about 8.5 PPM, at least about 8.6 PPM, at least about 8.7 PPM, at least about 8.8 PPM, at least about 8.9 PPM, at least about 9.0 PPM, at least about 9.1 PPM, at least about 9.2 PPM, at least about 9.3 PPM, at least about 9.4 PPM, at least about 9.5 PPM, at least about 9.6 PPM, at least about 9.7 PPM, at least about 9.8 PPM, at least about 9.9 PPM, at least about 10.0 PPM, at least about 10.1 PPM, at least about 10.2 PPM, at least about 10.3 PPM, at least about 10.4 PPM, at least about 10.5 PPM, at least about 10.6 PPM, at least about 10.7 PPM, at least about 10.8 PPM, at least about 10.9 PPM, at least about 11.0 PPM, or a range between any two of these values.

In some embodiments, the epicatechin present in the latex is in an amount of at least about 2.0 PPM, at least about 2.1 PPM, at least about 2.2 PPM, at least about 2.3 PPM, at least about 2.4 PPM, at least about 2.5 PPM, at least about 2.6 PPM, at least about 2.7 PPM, at least about 2.8 PPM, at least about 2.9 PPM, at least about 3.0 PPM, at least about 3.1 PPM, at least about 3.2 PPM, at least about 3.3 PPM, at least about 3.4 PPM, at least about 3.5 PPM, at least about 3.6 PPM, at least about 3.7 PPM, at least about 3.8 PPM, at least about 3.9 PPM, at least about 4.0 PPM, at least about 4.1 PPM, at least about 4.2 PPM, at least about 4.3 PPM, at least about 4.4 PPM, at least about 4.5 PPM, at least about 4.6 PPM, at least about 4.7 PPM, at least about 4.8 PPM, at least about 4.9 PPM, at least about 5.0 PPM, at least about 5.1 PPM, at least about 5.2 PPM, at least about 5.3 PPM, at least about 5.4 PPM, at least about 5.5 PPM, at least about 5.6 PPM, at least about 5.7 PPM, at least about 5.8 PPM, at least about 5.9 PPM, at least about 6.0 PPM, at least about 6.1 PPM, at least about 6.2 PPM, at least about 6.3 PPM, at least about 6.4 PPM, at least about 6.5 PPM, at least about 6.6 PPM, at least about 6.7 PPM, at least about 6.8 PPM, at least about 6.9 PPM, at least about 7.0 PPM, at least about 7.1 PPM, at least about 7.2 PPM, at least about 7.3 PPM, at least about 7.4 PPM, at least about 7.5 PPM, at least about 7.6 PPM, at least about 7.7 PPM, at least about 7.8 PPM, at least about 7.9 PPM, at least about 8.0 PPM, at least about 8.1 PPM, at least about 8.2 PPM, at least about 8.3 PPM, at least about 8.4 PPM, at least about 8.5 PPM, at least about 8.6 PPM, at least about 8.7 PPM, at least about 8.8 PPM, at least about 8.9 PPM, at least about 9.0 PPM, at least about 9.1 PPM, at least about 9.2 PPM, at least about 9.3 PPM, at least about 9.4 PPM, at least about 9.5 PPM, at least about 9.6 PPM, at least about 9.7 PPM, at least about 9.8 PPM, at least about 9.9 PPM, at least about 10.0 PPM, or a range between any two of these values.

In some embodiments, the taspine present in the latex is in an amount of at least about 45 PPM, at least about 46 PPM, at least about 47 PPM, at least about 48 PPM, at least about 49 PPM, at least about 50 PPM, at least about 51 PPM, at least about 52 PPM, at least about 53 PPM, at least about 54 PPM, at least about 55 PPM, at least about 56 PPM, at least about 57 PPM, at least about 58 PPM, at least about 59 PPM, at least about 60 PPM, at least about 61 PPM, at least about 62 PPM, at least about 63 PPM, at least about 64 PPM, at least about 65 PPM, or a range between any two of these values.

In some embodiments, the dimethylcedrusin present in the latex is in an amount of at least about 0.1 mg of dimethylcedrusin/kg of latex, at least about 0.11 PPM, at least about 0.12 PPM, at least about 0.13 PPM, at least about 0.14 PPM, at least about 0.15 PPM, at least about 0.16 PPM, at least about 0.17 PPM, at least about 0.18 PPM, at least about 0.18 PPM, at least about 0.19 PPM, at least about 0.20 PPM, at least about 0.21 PPM, at least about 0.22 PPM, at least about 0.23 PPM, at least about 0.24 PPM, at least about 0.25 PPM, at least about 0.26 PPM, at least about 0.27 PPM, at least about 0.28 PPM, at least about 0.29 PPM, at least about 0.30 PPM, at least about 0.31 PPM, at least about 0.32 PPM, at least about 0.33 PPM, at least about 0.34 PPM, at least about 0.35 PPM, at least about 0.36 PPM, at least about 0.37 PPM, at least about 0.38 PPM, at least about 0.39 PPM, about 0.40 PPM, at least about 0.41 PPM, at least about 0.42 PPM, at least about 0.43 PPM, at least about 0.44 PPM, at least about 0.45 PPM, at least about 0.46 PPM, at least about 0.47 PPM, at least about 0.48 PPM, at least about 0.49 PPM, at least about 0.5 PPM, at least about 0.6 PPM, at least about 0.7 PPM, at least about 0.8 PPM, at least about 0.9 PPM, at least about 1.0 PPM, at least about 1.1 PPM, at least about 1.2 PPM, at least about 1.3 PPM, at least about 1.4 PPM, at least about 1.5 PPM, at least about 1.6 PPM, at least about 1.7 PPM, at least about 1.8 PPM, at least about 1.9 PPM, at least about 2.0 PPM, at least about 2.1 PPM, at least about 2.2 PPM, at least about 2.3 PPM, at least about 2.4 PPM, at least about 2.5 PPM, at least about 2.6 PPM, at least about 2.7 PPM, at least about 2.8 PPM, at least about 2.9 PPM, at least about 3.0 PPM, at least about 3.1 PPM, at least about 3.2 PPM, at least about 3.3 PPM, at least about 3.4 PPM, at least about 3.5 PPM, at least about 3.6 PPM, at least about 3.7 PPM, at least about 3.8 PPM, at least about 3.9 PPM, at least about 4.0 PPM, at least about 4.1 PPM, at least about 4.2 PPM, at least about 4.3 PPM, at least about 4.4 PPM, at least about 4.5 PPM, at least about 4.6 PPM, at least about 4.7 PPM, at least about 4.8 PPM, at least about 4.9 PPM, at least about 5.0 PPM, at least about 5.1 PPM, at least about 5.2 PPM, at least about 5.3 PPM, at least about 5.4 PPM, at least about 5.5 PPM, at least about 5.6 PPM, at least about 5.7 PPM, at least about 5.8 PPM, at least about 5.9 PPM, at least about 6.0 PPM, at least about 6.1 PPM, at least about 6.2 PPM, at least about 6.3 PPM, at least about 6.4 PPM, at least about 6.5 PPM, at least about 6.6 PPM, at least about 6.7 PPM, at least about 6.8 PPM, at least about 6.9 PPM, at least about 7.0 PPM, at least about 7.1 PPM, at least about 7.2 PPM, at least about 7.3 PPM, at least about 7.4 PPM, at least about 7.5 PPM, at least about 7.6 PPM, at least about 7.7 PPM, at least about 7.8 PPM, at least about 7.9 PPM, at least about 8.0 PPM, at least about 8.1 PPM, at least about 8.2 PPM, at least about 8.3 PPM, at least about 8.4 PPM, at least about 8.5 PPM, at least about 8.6 PPM, at least about 8.7 PPM, at least about 8.8 PPM, at least about 8.9 PPM, at least about 9.0 PPM, at least about 9.1 PPM, at least about 9.2 PPM, at least about 9.3 PPM, at least about 9.4 PPM, at least about 9.5 PPM, at least about 9.6 PPM, at least about 9.7 PPM, at least about 9.8 PPM, at least about 9.9 PPM, at least about 10.0 PPM, or a range between any two of these values.

Polydispersity Index (PDI) is used to measure the breadth of the molecular weight distribution of AB-101. PDI is used to indicate distribution of polymer chain molecular weights in a given polymer, as the PDI value increases the heterogeneity in cross-linking, network formation, chain length, branching, hyper branching is increased and will have a more random arrangement. PDI is an important measure to characterize the unique compositional nature of AB-101 and other Croton lechleri derived compositions.

In any of the embodiments disclosed herein, the latex of Croton lechleri, preferably filtered latex of Croton lechleri, preferably filtered latex of Croton lechleri Müll.Arg has a PDI of about 0.5 to 0.85, about 0.55 to 0.85, about 0.6 to 0.85, about 0.65 to 0.85, about 0.7 to 0.85, about 0.75 to 0.85, about 0.8 to 0.85, about 0.5 to about 0.8, about 0.5 to about 0.75, about 0.5 to about 0.7, about 0.5 to about 0.65, about 0.5 to about 0.6, 0.5 to about 0.55, or a value within one of these ranges. Specific examples may include about 0.5, about 0.55, about 0.6, about 0.65, about 0.7, about 0.75, about 0.8, about 0.85, or a range between any two of these values. In some embodiments, the latex of Croton lechleri, preferably filtered latex of Croton lechleri, preferably filtered latex of Croton lechleri Müll.Arg has a PDI of about 0.81.

The pharmaceutical composition of AB-101 as described and claimed herein is a plant sourced material that meets the criteria of being consistently reproducible between batch to batch and reliably delivers the desired health benefits via oral inhalation and/or nasal inhalation that may be used in a pharmaceutical composition. It can be used to treat cystic fibrosis and/or a bacterial infection in a cystic fibrosis patient. Plant sourced materials face the challenge that changes in environmental weather, climate, rainfall, time of harvest (via season, time of day or month), changes in geography, longitude location, latitude location, altitude, changes in soil condition, harvesting protocols and many additional conditions can alter the characteristics of the plant that could impact quality. This can impact the plant's bioactivity resulting in inconsistency in achieving desired performance outcome. This creates a challenge in defining a pharmaceutical grade of dragon's blood to deliver consistent and reproducible therapeutic benefits. This is further compounded by the wide variety of the different species called dragon's blood. For example, phytochemical and anti-staphylococcal biofilm assessment of Dracaena draco L. Spp. draco resin, referred as dragon's blood, is “inactive in the maximum tested concentration of 1000 mcg/ml against free living staphylococci.” In contrast, AB-101 (latex of Croton lechleri, preferably filtered latex of Croton lechleri, preferably filtered latex of Croton lechleri Müll.Arg. where the components that have a negative impact on the delivery and/or treatment via inhalation have optionally been removed with the appropriate levels of gallocatechin, epigallocatechin, catechin, epicatechin, taspine, and dimethylcedrusin) is effective against Staphylococcus specifically methicillin-susceptible Staphylococcus aureus (MSSA) or the shorten nomenclature staph bacteria and in particular methicillin-resistant Staphylococcus aureus (MRSA) and in particular Mupirocin resistant MRSA. The generic name of the Croton lechleri resin, ie, dragon's blood, or Sangre de grado, creates confusion in defining a plant-derived pharmaceutical and demonstrates that not all Croton lechleri plants are the same, nor do they provide similar benefits.

The benefits of AB-101, filtered or unfiltered latex of Croton lechleri, preferably filtered latex of Croton lechleri, preferably filtered latex of Croton lechleri Müll.Arg., where the components that have a negative impact on the delivery and/or treatment via inhalation have optionally been removed is its ability to deliver consistent results for treating the pathogens between batch to batch in spite of all the confounding conditions. The challenge in using the whole latex is to identify the compounds that deliver performance based on the many bio-active compounds comprising the latex. Even within the same species, grown in a similar location, there are variations in chemical content and bioactivity of the whole latex that unexpectedly varies in its ability to fight and kill pathogens.

Methodology that can identify the whole latex is effective by having an assay that determines when a batch meets the predetermined performance criteria. Having a unique analytical and microbiological assay enables the ability to identify which batch of filtered or unfiltered latex of Croton lechleri, preferably filtered latex of Croton lechleri, preferably filtered latex of Croton lechleri Müll.Arg, where the components that have a negative impact on the delivery and/or treatment via inhalation have optionally been removed has the combination of components that will consistently deliver the desired outcome.

AB-101 botanical raw material (BRM) is a complex botanical product that is a latex of Croton lechleri, preferably filtered latex of Croton lechleri, preferably filtered latex of Croton lechleri Müll.Arg. where the components that have a negative impact on the delivery and/or treatment via inhalation have optionally been removed that contains certain marker compounds (catechin, gallocatechin, epicatechin, epigallocatechin, taspine, and dimethylcedrusin) in specified amounts (see Table 1a). Utilization of liquid chromatography with tandem mass spectrometry (LC-MS/MS) can be used to characterize the existence and levels of such marker compounds for batch to batch consistency and repeatable performance of AB-101. Marker compounds in AB-101 BRM include the proanthocyanidins: catechin, gallocatechin, epicatechin, and epigallocatechin, the alkaloid taspine, as well as dimethylcedrusin.

The published and accepted taxonomic classification of Croton lechleri Müll.Arg. is the following (van Ee & Berry, 2011, Riina et al, 2009, The Plant List, 2012, The Angiosperm Phylogeny Group, 2009):

### Division: Streptophyta

Class: Equisetopsida
Subclass: Magnoliidae
   Order: Malpighiales
      Family: Euphorbiaceae
         Genus: Croton
         Subgenus Adenophylli
         Section: Cyclostigma
         Subsection: Cyclostigma
         Species: Croton lechleri Müll.Arg.

Biodiversity of botanicals plays a major role in constituent chemical compound characterization. Chemical compounds utilized for as important batch to batch consistency of AB-101 need to 1) demonstrate antimicrobial or cicatrizant properties, 2) be present in AB-101, and 3) be detectable using analytical techniques. Using these criteria, the analytical efforts focused on 3 classes of compounds: polyphenols (proanthocyanidins), alkaloids (taspine), and lignin (dimethylcedrusin). Within the proanthocyanidin class, 4 specific compounds were focused on: catechin, epicatechin, gallocatechin, and epigallocatechin. The compound of importance within the alkaloid class is taspine. Finally, the compound of importance within the lignin class is dimethylcedrusin. Each of these compounds fulfills the three required elements detailed above. The following are the chemical structures of the 6 compounds utilized as important markers for batch to batch consistency of AB-101.

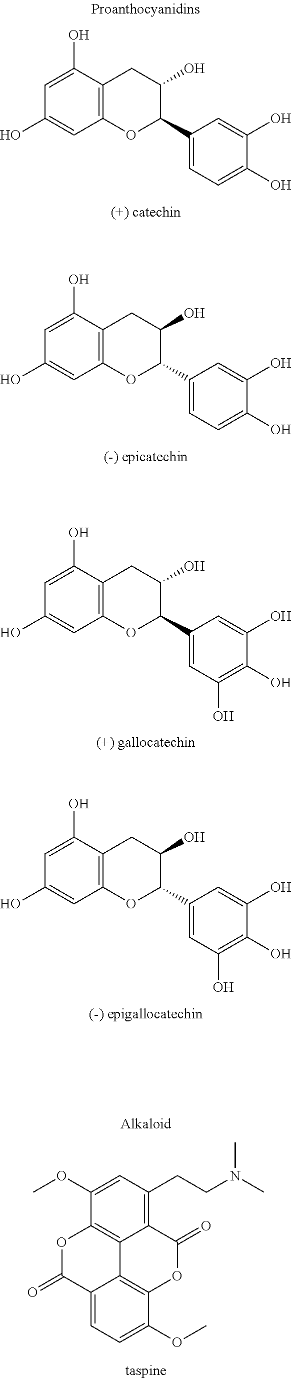 [SMILES: Oc1cc(O)c2c(c1)O[C@H](c1ccc(O)c(O)c1)[C@@H](O)C2 ; Oc1cc(O)c2c(c1)O[C@@H](c1ccc(O)c(O)c1)[C@H](O)C2 ; Oc1cc(O)c2c(c1)O[C@H](c1cc(O)c(O)c(O)c1)[C@@H](O)C2 ; Oc1cc(O)c2c(c1)O[C@@H](c1cc(O)c(O)c(O)c1)[C@H](O)C2 ; COc1ccc2c(=O)oc3c(OC)cc(CCN(C)C)c4c(=O)oc1c2c34]

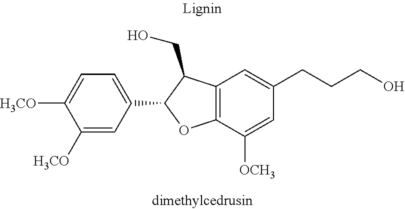 [SMILES: COc1ccc([C@H]2Oc3c(OC)cc(CCCO)cc3[C@@H]2CO)cc1OC]

For characterization studies, AB-101 extract was lyophilized and the lyophilized powder was subjected to three different extraction methods.

Method 1—Ultrasonic polyphenol extraction. The lyophilized AB-101 extract was dissolved into methanol. The resultant emulsion was then subjected to sonication for 10 minutes followed by centrifugation to remove particulates for 5 minutes. The supernatant was then subjected to LC-MS/MS analysis.

Method 2—Soxhlet extraction. The lyophilized AB-101 extract was subjected to a Soxhlet extraction with 80% ethanol. The ethanol was removed via a rotary evaporator. The resultant material was then subjected re-suspended in ethanol then subjected to LC-MS/MS analysis.

Method 3—Polyphenol extraction. The lyophilized AB-101 extract was incubated with methanol overnight at room temperature and in the dark. The supernatant was then filtered using Whatman filters, dried, and then re-suspended in methanol. The resultant material was then subjected to LC-MS/MS analysis.

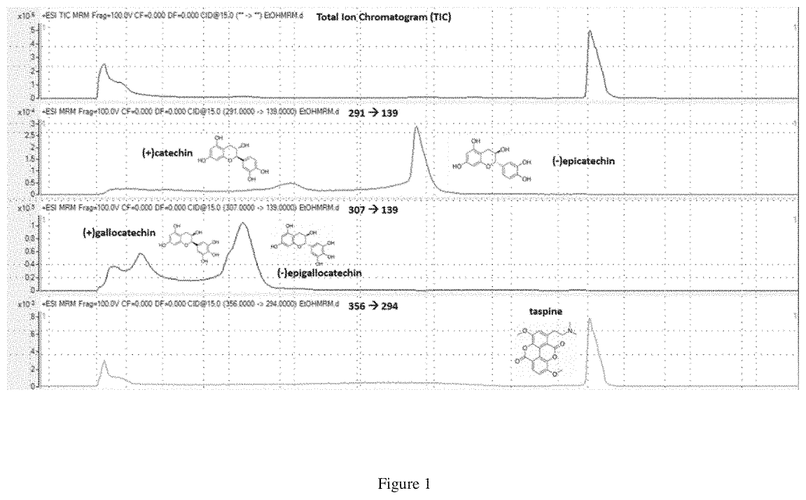

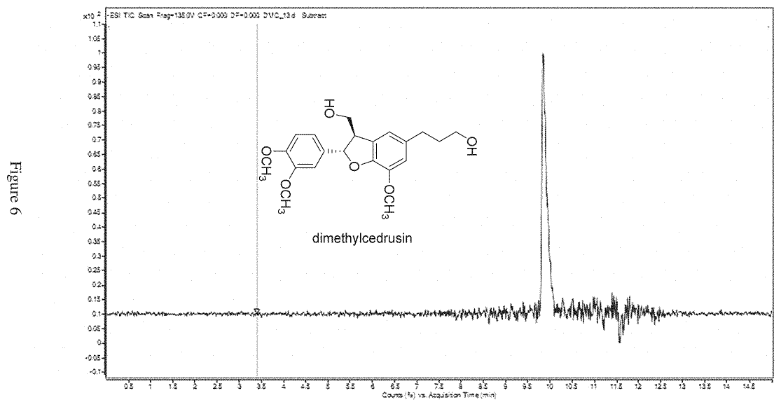

FIG. 1 depicts a representative Total Ion Chromatogram as well as additional Multiple Reaction Monitoring spectra that identify the important marker compounds in an AB-101 extract. While FIG. 6 depicts a representative Total Ion Chromatogram of dimethylcedrusin. The compounds are detectable using any of the three extraction methods.

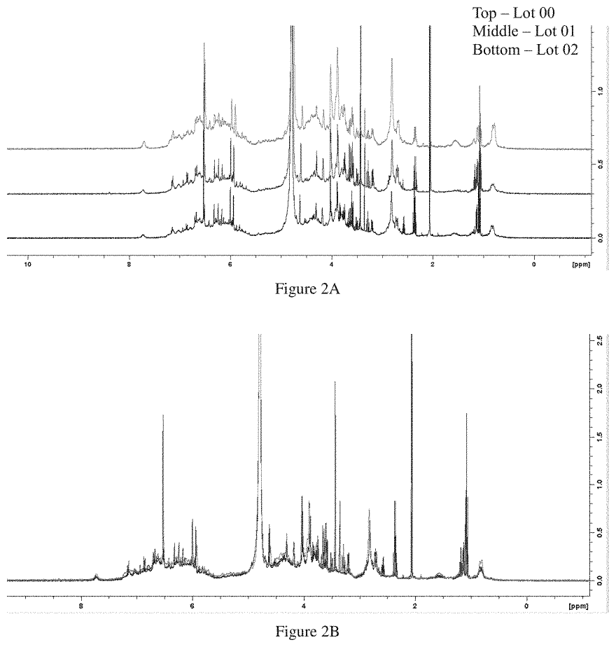

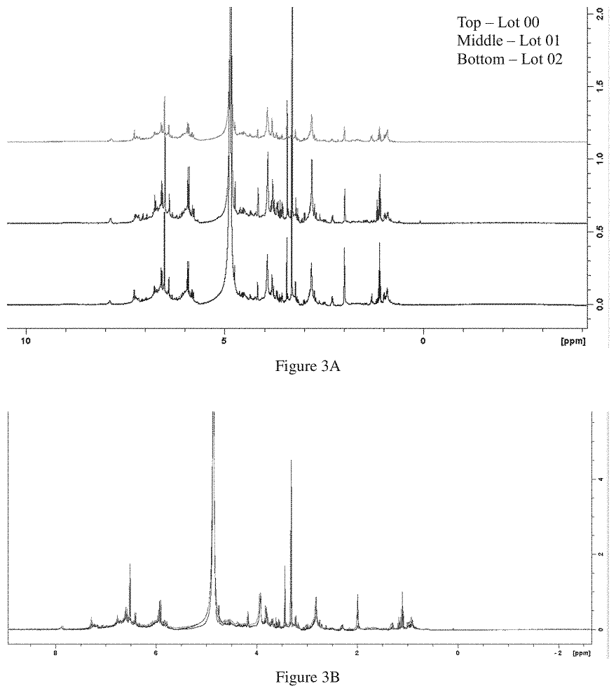

Biodiversity contributes to vast amounts of variability. In order to capture this variability, an NMR method utilizing a “spectral fingerprint” was used with an overlapping a reference standard. These fingerprinting captures most components within AB-101 and would be quantifiable using Nuclear Magnetic Resonance (NMR). Examples of NMR spectra using three different AB-101 lots (Lots 00, 01, and 02 respectively) and two different deuterated solvents (D₂O and d₄-Methanol respectively) are shown in FIGS. 2A and 3A with overlays of each solvents spectra being shown in FIGS. 2B and 3B and demonstrated no significant variability.

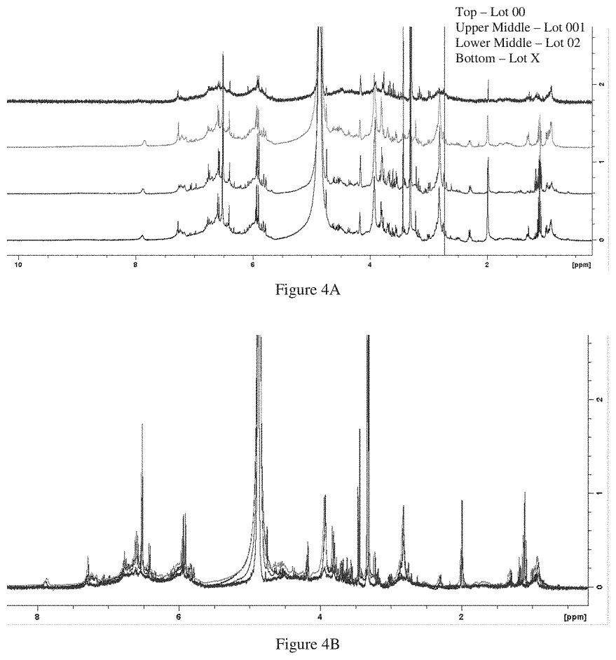

In another NMR analysis using the d₄-Methanol as the solvent, 4 distinct lots of AB-101 (Lots 00, 01, 02, and X respectively) are compared. NMR spectra of each lot are shown in FIG. 4A with overlays of each lots spectra being shown in FIG. 4B. While the fingerprint of the 4 lots looks similar, there are important differences. This is shown by comparing the concentration level in ppm based on LC-MS/MS Quantification and qualitative NMR “fingerprinting” on the marker compounds of catechin, epicatechin, gallocatechin, epigallocatechin, taspine, and dimethylcedrusin. The results are shown in Table 2 and indicate that lots 1 and 2 are more similar and lots X and 0 have the largest differences.

TABLE 2

AB-101 Lots Characterization
PPM (μg/g)

| Lot | X | 00 | 01 | 02 |
|---|---|---|---|---|
| Gallocatechin (GC) | 164.2 | 91.9 | 135.0 | 139.9 |
| Epigallocatechin (EGC) | 1357.6 | 380.7 | 1219.5 | 996.3 |
| Catechin (C) | 2.0 | 6.7 | 8.8 | 8.2 |
| Epicatechin (EC) | 2.6 | 5.2 | 8.3 | 6.1 |
| Taspine (T) | 50.4 | 43.4 | 50.1 | 51.1 |
| Dimethylcedrusin | 0.1 | 0.1 | 0.1 | 0.1 |

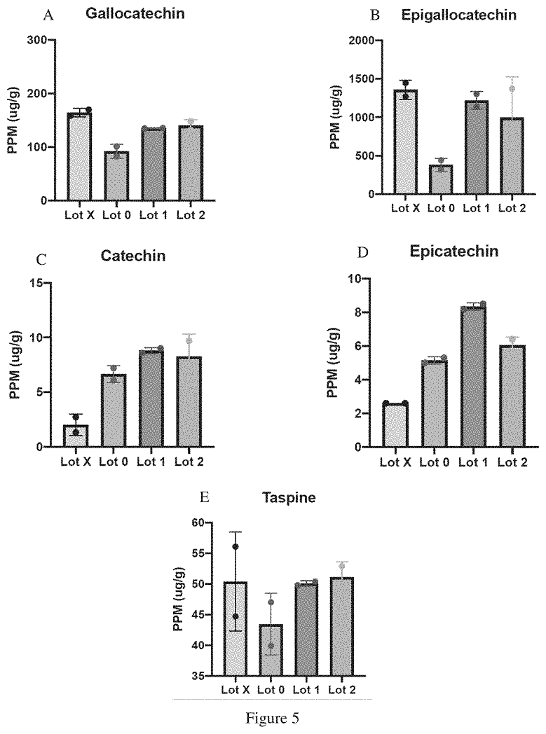

FIG. 5A-E depicts bar graphs comparing the AB-101 lot analysis results for each of the 5 marker compounds.

Lot 00 is an example of a lot that is not suitable for use in the pharmaceutical compositions and the methods of use described herein. Lots X, 01 and 02 are examples of lots that are suitable for use in the pharmaceutical compositions and the methods of use described herein.

Zheng-Ping Chen publication (Studies on the Anti-Tumour, Anti-Bacterial and Wound-Healing Properties of Dragon's Blood, Planta Med. 60 (1994)) demonstrates the non-obviousness of identifying the optimum properties of pharmaceutical grade AB-101. Chen uses a bioassay used to measure the incorporation rate of H-thymidine into the DNA of the cells in the presence of the test sample. This bioassay provides a measure of the wound healing property of the “sap.” Chen uses the Croton lechleri Müll.Arg latex from Ecuador. This assay indicated that the dried sap and MeOH Extract would have an incorporation rate of 68+/−12 and 88=/−5. According to Chen the dried sap and MeOH was found to be very inhibitory to wound healing properties. One familiar in the art would not assume that a Croton lechleri Müll.Arg latex extract as a whole would be effective in wound healing properties. Further, Chen states that the Ecuador sap contained only traces of taspine. Chen wanted to completely minimize or eliminate taspine due to the concern of being cytotoxin. Chen evaluated specific compound through extraction. Specifically, in the case of gallocatechin and epigalocatechin were rated as slightly stimulating to cell proliferation, while Catechin and Epicatechin showed little effect. Further Chen states that taspine and dimethylcedrusin showed little healing effects in the Ecuadorian sap.

The pharmaceutical grade of AB-101 identified a unique composition to maximize the healing properties while maintaining the film forming, low Log P and antibiotic activity. While Chen would not use the whole Croton lechleri Müll.Arg latex containing taspine or dimethylcedrusin, AB-101 pharmaceutical grade maintained using the entire Croton lechleri Müll.Arg latex in the composition for medicinal benefits associated with treating cystic fibrosis and/or a bacterial infection in a cystic fibrosis patient. Taspine has antibiotic, antiviral and anti-inflammatory properties. Dimethylcedrusin has unique fibroblast stimulating properties to promote healing. Taspine was targeted at least about 45 PPM and dimethylcedrusin was targeted to have a detectable presence be at least about 0.1 PPM. gallocatechin and epigallocatechin were optimized to have a combined total composition of at least about 60% of the total 4 catechins where epigallocatechin was to have a composition at least about 45% of the total 4 catechins.

In some embodiments the latex of Croton lechleri, preferably filtered latex of Croton lechleri, preferably filtered latex of Croton lechleri Müll.Arg. where the components that have a negative impact on the delivery and/or treatment via inhalation have optionally been removed has a minimum bactericidal concentration (MBC) of about 6.25 (% vol./vol.), about 12.5 (% vol./vol.), about 25 (% vol./vol.), about 50 (% vol./vol.), or a range between any two of these values. In some embodiments the latex of Croton lechleri, preferably filtered latex of Croton lechleri, preferably filtered latex of Croton lechleri Müll.Arg. where the components that have a negative impact on the delivery and/or treatment via inhalation have optionally been removed has a MBC of about 6.25 (% vol./vol.) to about 50(% vol./vol.).

The pharmaceutical composition, as used in the embodiments described herein, is suitable for administration via inhalation. As such, the latex of Croton lechleri, preferably filtered latex of Croton lechleri, preferably filtered latex of Croton lechleri Müll.Arg. is devoid of any components and/or substances that would negatively impact administration via inhalation. As an example, devoid of The components that could impact inhalation include the removal of organic compound fractions and components that can coat the lungs and limit lung function, preventing the absorption of oxygen, and particulate matter of the latex that may be irritating to the lungs consistent with the Environmental Protection Agencies guidance stating “The size of particles is directly linked to their potential for causing health problems. Small particles less than 10 micrometers in diameter pose the greatest problems, because they can get deep into your lungs, and some may even get into your bloodstream. (https://www.epa.gov/pm-pollution/health-and-environmental-effects-particulate-matter-pm).

The excipient(s) must be “acceptable” in the sense of being compatible with the other ingredients of the formulation and not deleterious to the recipient thereof. The excipient(s) will utilize a low number of known, well-characterized excipient ingredients that will not impart irritation or sensitization when used in oral inhalation and/or nasal inhalation or reduce the efficacy of AB-101. Proper formulation of the pharmaceutical composition is dependent upon the route of administration chosen. Any of the well-known techniques and excipients may be used as suitable and as understood in the art. The pharmaceutical compositions disclosed herein may be manufactured in any manner known in the art.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose, including eutectic solvents, eutectic-based ionic liquids, or ionic liquids. The pharmaceutical compositions can additionally include: lubricating agents such as talc, magnesium stearate, and mineral oil; wetting agents; emulsifying and suspending agents; preserving agents such as methyl- and propylhydroxy-benzoates.

The compositions include those suitable for administration to the lungs (including, for example, oral inhalation and/or nasal inhalation). The compositions may conveniently be presented in unit dosage form and may be prepared by any of the methods well known in the art of pharmacy. Typically, these methods include the step of bringing into association latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the composition contains at least the concentration of components of latex of Croton lechleri, preferably the concentration of components of filtered latex of Croton lechleri, preferably the concentration of components of filtered latex of Croton lechleri Müll.Arg of the reference standard, disclosed herein (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired composition.

The pharmaceutical compositions disclosed herein may be administered to the lungs, that is by non-systemic administration. In embodiments, such administration or an orally inhalable and/or nasally inhalable pharmaceutical composition does not result in systemic administration or systemic exposure of the Croton lechleri to the patient.

In some embodiments, pharmaceutical compositions suitable for administration to the lungs include liquid or semi-liquid preparations suitable for oral inhalation and/or nasal inhalation such as a solution, powder, fluid emulsion, fluid suspension, lotion, and drops.

Preferred unit dosage pharmaceutical compositions are those containing an effective dose, as herein below recited, or an appropriate fraction thereof, of the active ingredient.

When employed as pharmaceuticals, the compounds can be administered in the form of pharmaceutical compositions. These compositions can be prepared in a manner well known in the pharmaceutical arts, and can be administered by a variety of routes, depending upon whether local or systemic treatment is desired and upon the area to be treated. Administration of the disclosed compounds or compositions may be inhaled (including oral inhalation and/or nasal inhalation). Pharmaceutical compositions for administration to the lungs may include solutions, fluid emulsions, fluid suspensions, semi-solids, aerosols, sprays, liquids, aerosolization, inhalers, and powders. Conventional pharmaceutical carriers, aqueous, powder or oily bases, thickeners and the like may be necessary or desirable. In some embodiments, the compounds can be contained in such pharmaceutical compositions with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The artisan can refer to various pharmacologic references for guidance.

In certain embodiments, the pharmaceutical composition is a liquid.

In certain embodiments, the pharmaceutical composition is a powder.

The pharmaceutical compositions can be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, active pharmaceutical compositions comprising latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the composition contains at least the concentration of components of latex of Croton lechleri, preferably the concentration of components of filtered latex of Croton lechleri, preferably the concentration of components of filtered latex of Croton lechleri Müll.Arg of the reference standard, can be formulated for administration to the lungs via oral inhalation and/or nasal inhalation.

The active pharmaceutical compositions comprising latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the composition contains at least the concentration of components of latex of Croton lechleri, preferably the concentration of components of filtered latex of Croton lechleri, preferably the concentration of components of filtered latex of Croton lechleri Müll.Arg of the reference standard, can be effective over a wide dosage range and can be generally administered in a therapeutically effective amount. It will be understood, however, that the amount of the pharmaceutical compositions comprising latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the composition contains at least the concentration of components of latex of Croton lechleri, preferably the concentration of components of filtered latex of Croton lechleri, preferably the concentration of components of filtered latex of Croton lechleri Müll.Arg of the reference standard, actually administered will usually be determined by a physician, according to the relevant circumstances, including the condition to be treated, the chosen route of administration, the actual compound administered, the age, weight, and response of the individual patient, the severity of the patient's symptoms, and the like.

In some embodiments, the pharmaceutical composition may comprise about 0.1 wt % to about 60 wt % of latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the composition contains at least the concentration of components of latex of Croton lechleri, preferably the concentration of components of filtered latex of Croton lechleri, preferably the concentration of components of filtered latex of Croton lechleri Müll.Arg of the reference standard, disclosed herein. In some embodiments, the latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the composition contains at least the concentration of components of latex of Croton lechleri, preferably the concentration of components of filtered latex of Croton lechleri, preferably the concentration of components of filtered latex of Croton lechleri Müll.Arg of the reference standard, is in an amount of about 0.1 wt % to about 99 wt %, about 0.1 wt % to about 98 wt %, about 0.1 wt % to about 97 wt %, about 0.1 wt % to about 96 wt %, about 0.1 wt % to about 95 wt %, about 0.1 wt % to about 90 wt %, about 0.1 wt % to about 85 wt %, about 0.1 wt % to about 80 wt %, about 0.1 wt % to about 75 wt %, about 0.1 wt % to about 70 wt %, about 0.1 wt % to about 65 wt %, about 0.1 wt % to about 55 wt %, about 0.1 wt % to about 50 wt %, about 0.1 wt % to about 45 wt %, about 0.1 wt % to about 40 wt %, about 0.1 wt % to about 30 wt %, about 0.1 wt % to about 20 wt %, about 0.1 wt % to about 10 wt %, about 0.1 wt % to about 5 wt %, about 0.5 wt % to about 50 wt %, about 0.5 wt % to about 45 wt %, about 0.5 wt % to about 40 wt %, about 0.5 wt % to about 30 wt %, about 0.5 wt % to about 20 wt %, about 0.5 wt % to about 10 wt %, about 0.5 wt % to about 5 wt %, about 1 wt % to about 300 wt %, about 1 wt % to about 295 wt %, about 1 wt % to about 290 wt %, about 1 wt % to about 285 wt %, about 1 wt % to about 280 wt %, about 1 wt % to about 275 wt %, about 1 wt % to about 270 wt %, about 1 wt % to about 265 wt %, about 1 wt % to about 260 wt %, about 1 wt % to about 255 wt %, about 1 wt % to about 250 wt %, about 1 wt % to about 245 wt %, about 1 wt % to about 240 wt %, about 1 wt % to about 235 wt %, about 1 wt % to about 230 wt %, about 1 wt % to about 225 wt %, about 1 wt % to about 220 wt %, about 1 wt % to about 215 wt %, about 1 wt % to about 210 wt %, about 1 wt % to about 205 wt %, about 1 wt % to about 200 wt %, 195 wt %, about 1 wt % to about 190 wt %, about 1 wt % to about 185 wt %, about 1 wt % to about 180 wt %, about 1 wt % to about 175 wt %, about 1 wt % to about 170 wt %, about 1 wt % to about 165 wt %, about 1 wt % to about 160 wt %, about 1 wt % to about 155 wt %, about 1 wt % to about 150 wt %, about 1 wt % to about 145 wt %, about 1 wt % to about 140 wt %, about 1 wt % to about 135 wt %, about 1 wt % to about 130 wt %, about 1 wt % to about 125 wt %, about 1 wt % to about 120 wt %, about 1 wt % to about 115 wt %, about 1 wt % to about 10 wt %, about 1 wt % to about 105 wt %, about 1 wt % to about 100 wt %, about 1 wt % to about 95 wt %, about 1 wt % to about 90 wt %, about 1 wt % to about 85 wt %, about 1 wt % to about 80 wt %, about 1 wt % to about 75 wt %, about 1 wt % to about 70 wt %, about 1 wt % to about 65 wt %, about 1 wt % to about 60 wt %, about 1 wt % to about 55 wt %, about 1 wt % to about 50 wt %, about 1 wt % to about 45 wt %, about 1 wt % to about 40 wt %, about 1 wt % to about 35 wt %, about 1 wt % to about 30 wt %, about 1 wt % to about 25 wt %, about 1 wt % to about 20 wt %, about 1 wt % to about 15 wt %, about 1 wt % to about 10 wt %, about 1 wt % to about 5 wt %, about 5 wt % to about 45 wt %, about 5 wt % to about 40 wt %, about 5 wt % to about 35 wt %, about 5 wt % to about 30 wt %, about 5 wt % to about 25 wt %, about 5 wt % to about 20 wt %, about 5 wt % to about 15 wt %, about 5 wt % to about 10 wt %, about 2 wt % to about 300 wt %, about 2 wt % to about 295 wt %, about 2 wt % to about 290 wt %, about 2 wt % to about 285 wt %, about 2 wt % to about 280 wt %, about 2 wt % to about 275 wt %, about 2 wt % to about 270 wt %, about 2 wt % to about 265 wt %, about 2 wt % to about 260 wt %, about 2 wt % to about 255 wt %, about 2 wt % to about 250 wt %, about 2 wt % to about 245 wt %, about 2 wt % to about 240 wt %, about 2 wt % to about 235 wt %, about 2 wt % to about 230 wt %, about 2 wt % to about 225 wt %, about 2 wt % to about 220 wt %, about 2 wt % to about 215 wt %, about 2 wt % to about 210 wt %, about 2 wt % to about 205 wt %, about 2 wt % to about 200 wt %, 195 wt %, about 2 wt % to about 190 wt %, about 2 wt % to about 185 wt %, about 2 wt % to about 180 wt %, about 2 wt % to about 175 wt %, about 2 wt % to about 170 wt %, about 2 wt % to about 165 wt %, about 2 wt % to about 160 wt %, about 2 wt % to about 155 wt %, about 2 wt % to about 150 wt %, about 2 wt % to about 145 wt %, about 2 wt % to about 140 wt %, about 2 wt % to about 135 wt %, about 2 wt % to about 130 wt %, about 2 wt % to about 125 wt %, about 2 wt % to about 120 wt %, about 2 wt % to about 115 wt %, about 2 wt % to about 10 wt %, about 2 wt % to about 105 wt %, about 2 wt % to about 100 wt %, about 2 wt % to about 95 wt %, about 2 wt % to about 90 wt %, about 2 wt % to about 85 wt %, about 2 wt % to about 80 wt %, about 2 wt % to about 75 wt %, about 2 wt % to about 70 wt %, about 2 wt % to about 65 wt %, about 2 wt % to about 60 wt %, about 2 wt % to about 55 wt %, about 2 wt % to about 50 wt %, about 2 wt % to about 45 wt %, about 2 wt % to about 40 wt %, about 2 wt % to about 35 wt %, about 2 wt % to about 30 wt %, about 2 wt % to about 25 wt %, about 2 wt % to about 20 wt %, about 2 wt % to about 15 wt %, about 3 wt % to about 300 wt %, about 3 wt % to about 295 wt %, about 3 wt % to about 290 wt %, about 3 wt % to about 285 wt %, about 3 wt % to about 280 wt %, about 3 wt % to about 275 wt %, about 3 wt % to about 270 wt %, about 3 wt % to about 265 wt %, about 3 wt % to about 260 wt %, about 3 wt % to about 255 wt %, about 3 wt % to about 250 wt %, about 3 wt % to about 245 wt %, about 3 wt % to about 240 wt %, about 3 wt % to about 235 wt %, about 3 wt % to about 230 wt %, about 3 wt % to about 225 wt %, about 3 wt % to about 220 wt %, about 3 wt % to about 215 wt %, about 3 wt % to about 210 wt %, about 3 wt % to about 205 wt %, about 3 wt % to about 200 wt %, 195 wt %, about 3 wt % to about 190 wt %, about 3 wt % to about 185 wt %, about 3 wt % to about 180 wt %, about 3 wt % to about 175 wt %, about 3 wt % to about 170 wt %, about 3 wt % to about 165 wt %, about 3 wt % to about 160 wt %, about 3 wt % to about 155 wt %, about 3 wt % to about 150 wt %, about 3 wt % to about 145 wt %, about 3 wt % to about 140 wt %, about 3 wt % to about 135 wt %, about 3 wt % to about 130 wt %, about 3 wt % to about 125 wt %, about 3 wt % to about 120 wt %, about 3 wt % to about 115 wt %, about 3 wt % to about 110 wt %, about 3 wt % to about 105 wt %, about 3 wt % to about 100 wt %, about 3 wt % to about 95 wt %, about 3 wt % to about 90 wt %, about 3 wt % to about 85 wt %, about 3 wt % to about 80 wt %, about 3 wt % to about 75 wt %, about 3 wt % to about 70 wt %, about 3 wt % to about 65 wt %, about 3 wt % to about 60 wt %, about 3 wt % to about 55 wt %, about 3 wt % to about 50 wt %, about 3 wt % to about 45 wt %, about 3 wt % to about 40 wt %, about 3 wt % to about 35 wt %, about 3 wt % to about 30 wt %, about 3 wt % to about 25 wt %, about 3 wt % to about 20 wt %, about 3 wt % to about 15 wt %, about 10 wt % to about 45 wt %, about 10 wt % to about 40 wt %, about 10 wt % to about 35 wt %, about 10 wt % to about 30 wt %, about 10 wt % to about 25 wt %, about 10 wt % to about 20 wt %, about 10 wt % to about 15 wt %, or a value within one of these ranges. Specific examples may include about 0.01 wt %, about 0.05 wt %, about 0.1 wt %, about 0.25 wt %, about 0.5 wt %, about 0.75 wt %, about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 60 wt %, about 70 wt %, about 80 wt %, about 90 wt %, about 100 wt %, about 110 wt %, about 120 wt %, about 130 wt %, about 140 wt %, about 150 wt %, about 160 wt %, about 170 wt %, about 180 wt %, about 190 wt %, about 200 wt %, about 210 wt %, about 220 wt %, about 230 wt %, about 240 wt %, about 250 wt %, about 260 wt %, about 270 wt %, about 280 wt %, about 290 wt %, about 300 wt %, or a range between any two of these values. The forgoing percentages are relative to a composition made from AB-101 with exemplary amounts of the marker compounds present in the latex as disclosed in Table 1. To illustrate, a pharmaceutical composition comprising 100 wt % of AB-101 will contain at least about 110 PPM of gallocatechin, while a pharmaceutical composition comprising 200 wt % of AB-101 will contain at least about 220 PPM of gallocatechin. The foregoing all representing weight percentages of embodiments of the pharmaceutical compositions. In some embodiments, the pharmaceutical composition is suitable for administration to the lungs (including, for example, oral inhalation and/or nasal inhalation).

In some embodiments, the latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the composition contains at least the concentration of components of latex of Croton lechleri, preferably the concentration of components of filtered latex of Croton lechleri, preferably the concentration of components of filtered latex of Croton lechleri Müll.Arg of the reference standard, is in a therapeutically effective amount. In some embodiments, the therapeutically effective amount may be in an amount of about 0.1 wt % to about 100 wt %, about 0.1 wt % to about 95 wt %, about 0.1 wt % to about 90 wt %, about 0.1 wt % to about 85 wt %, about 0.1 wt % to about 80 wt %, about 0.1 wt % to about 75 wt %, about 0.1 wt % to about 70 wt %, about 0.1 wt % to about 65 wt %, about 0.1 wt % to about 60 wt %, about 0.1 wt % to about 55 wt %, about 0.1 wt % to about 50 wt %, about 0.1 wt % to about 45 wt %, about 0.1 wt % to about 40 wt %, about 0.1 wt % to about 30 wt %, about 0.1 wt % to about 20 wt %, about 0.1 wt % to about 10 wt %, about 0.1 wt % to about 5 wt %, about 0.5 wt % to about 50 wt %, about 0.5 wt % to about 45 wt %, about 0.5 wt % to about 40 wt %, about 0.5 wt % to about 30 wt %, about 0.5 wt % to about 20 wt %, about 0.5 wt % to about 10 wt %, about 0.5 wt % to about 5 wt %, about 1 wt % to about 300 wt %, about 1 wt % to about 295 wt %, about 1 wt % to about 290 wt %, about 1 wt % to about 285 wt %, about 1 wt % to about 280 wt %, about 1 wt % to about 275 wt %, about 1 wt % to about 270 wt %, about 1 wt % to about 265 wt %, about 1 wt % to about 260 wt %, about 1 wt % to about 255 wt %, about 1 wt % to about 250 wt %, about 1 wt % to about 245 wt %, about 1 wt % to about 240 wt %, about 1 wt % to about 235 wt %, about 1 wt % to about 230 wt %, about 1 wt % to about 225 wt %, about 1 wt % to about 220 wt %, about 1 wt % to about 215 wt %, about 1 wt % to about 210 wt %, about 1 wt % to about 205 wt %, about 1 wt % to about 200 wt %, 195 wt %, about 1 wt % to about 190 wt %, about 1 wt % to about 185 wt %, about 1 wt % to about 180 wt %, about 1 wt % to about 175 wt %, about 1 wt % to about 170 wt %, about 1 wt % to about 165 wt %, about 1 wt % to about 160 wt %, about 1 wt % to about 155 wt %, about 1 wt % to about 150 wt %, about 1 wt % to about 145 wt %, about 1 wt % to about 140 wt %, about 1 wt % to about 135 wt %, about 1 wt % to about 130 wt %, about 1 wt % to about 125 wt %, about 1 wt % to about 120 wt %, about 1 wt % to about 115 wt %, about 1 wt % to about 110 wt %, about 1 wt % to about 105 wt %, about 1 wt % to about 100 wt %, about 1 wt % to about 95 wt %, about 1 wt % to about 90 wt %, about 1 wt % to about 85 wt %, about 1 wt % to about 80 wt %, about 1 wt % to about 75 wt %, about 1 wt % to about 70 wt %, about 1 wt % to about 65 wt %, about 1 wt % to about 60 wt %, about 1 wt % to about 55 wt %, about 1 wt % to about 50 wt %, about 1 wt % to about 45 wt %, about 1 wt % to about 40 wt %, about 1 wt % to about 35 wt %, about 1 wt % to about 30 wt %, about 1 wt % to about 25 wt %, about 1 wt % to about 20 wt %, about 1 wt % to about 15 wt %, about 1 wt % to about 10 wt %, about 1 wt % to about 5 wt %, about 5 wt % to about 45 wt %, about 5 wt % to about 40 wt %, about 5 wt % to about 35 wt %, about 5 wt % to about 30 wt %, about 5 wt % to about 25 wt %, about 5 wt % to about 20 wt %, about 5 wt % to about 15 wt %, about 5 wt % to about 10 wt %, about 2 wt % to about 300 wt %, about 2 wt % to about 295 wt %, about 2 wt % to about 290 wt %, about 2 wt % to about 285 wt %, about 2 wt % to about 280 wt %, about 2 wt % to about 275 wt %, about 2 wt % to about 270 wt %, about 2 wt % to about 265 wt %, about 2 wt % to about 260 wt %, about 2 wt % to about 255 wt %, about 2 wt % to about 250 wt %, about 2 wt % to about 245 wt %, about 2 wt % to about 240 wt %, about 2 wt % to about 235 wt %, about 2 wt % to about 230 wt %, about 2 wt % to about 225 wt %, about 2 wt % to about 220 wt %, about 2 wt % to about 215 wt %, about 2 wt % to about 210 wt %, about 2 wt % to about 205 wt %, about 2 wt % to about 200 wt %, 195 wt %, about 2 wt % to about 190 wt %, about 2 wt % to about 185 wt %, about 2 wt % to about 180 wt %, about 2 wt % to about 175 wt %, about 2 wt % to about 170 wt %, about 2 wt % to about 165 wt %, about 2 wt % to about 160 wt %, about 2 wt % to about 155 wt %, about 2 wt % to about 150 wt %, about 2 wt % to about 145 wt %, about 2 wt % to about 140 wt %, about 2 wt % to about 135 wt %, about 2 wt % to about 130 wt %, about 2 wt % to about 125 wt %, about 2 wt % to about 120 wt %, about 2 wt % to about 115 wt %, about 2 wt % to about 110 wt %, about 2 wt % to about 105 wt %, about 2 wt % to about 100 wt %, about 2 wt % to about 95 wt %, about 2 wt % to about 90 wt %, about 2 wt % to about 85 wt %, about 2 wt % to about 80 wt %, about 2 wt % to about 75 wt %, about 2 wt % to about 70 wt %, about 2 wt % to about 65 wt %, about 2 wt % to about 60 wt %, about 2 wt % to about 55 wt %, about 2 wt % to about 50 wt %, about 2 wt % to about 45 wt %, about 2 wt % to about 40 wt %, about 2 wt % to about 35 wt %, about 2 wt % to about 30 wt %, about 2 wt % to about 25 wt %, about 2 wt % to about 20 wt %, about 2 wt % to about 15 wt %, about 3 wt % to about 300 wt %, about 3 wt % to about 295 wt %, about 3 wt % to about 290 wt %, about 3 wt % to about 285 wt %, about 3 wt % to about 280 wt %, about 3 wt % to about 275 wt %, about 3 wt % to about 270 wt %, about 3 wt % to about 265 wt %, about 3 wt % to about 260 wt %, about 3 wt % to about 255 wt %, about 3 wt % to about 250 wt %, about 3 wt % to about 245 wt %, about 3 wt % to about 240 wt %, about 3 wt % to about 235 wt %, about 3 wt % to about 230 wt %, about 3 wt % to about 225 wt %, about 3 wt % to about 220 wt %, about 3 wt % to about 215 wt %, about 3 wt % to about 210 wt %, about 3 wt % to about 205 wt %, about 3 wt % to about 200 wt %, 195 wt %, about 3 wt % to about 190 wt %, about 3 wt % to about 185 wt %, about 3 wt % to about 180 wt %, about 3 wt % to about 175 wt %, about 3 wt % to about 170 wt %, about 3 wt % to about 165 wt %, about 3 wt % to about 160 wt %, about 3 wt % to about 155 wt %, about 3 wt % to about 150 wt %, about 3 wt % to about 145 wt %, about 3 wt % to about 140 wt %, about 3 wt % to about 135 wt %, about 3 wt % to about 130 wt %, about 3 wt % to about 125 wt %, about 3 wt % to about 120 wt %, about 3 wt % to about 115 wt %, about 3 wt % to about 110 wt %, about 3 wt % to about 105 wt %, about 3 wt % to about 100 wt %, about 3 wt % to about 95 wt %, about 3 wt % to about 90 wt %, about 3 wt % to about 85 wt %, about 3 wt % to about 80 wt %, about 3 wt % to about 75 wt %, about 3 wt % to about 70 wt %, about 3 wt % to about 65 wt %, about 3 wt % to about 60 wt %, about 3 wt % to about 55 wt %, about 3 wt % to about 50 wt %, about 3 wt % to about 45 wt %, about 3 wt % to about 40 wt %, about 3 wt % to about 35 wt %, about 3 wt % to about 30 wt %, about 3 wt % to about 25 wt %, about 3 wt % to about 20 wt %, about 3 wt % to about 15 wt %, about 5 wt % to about 100 wt %, about 5 wt % to about 95 wt %, about 5 wt % to about 90 wt %, about 5 wt % to about 85 wt %, about 5 wt % to about 80 wt %, about 5 wt % to about 75 wt %, about 5 wt % to about 70 wt %, about 5 wt % to about 65 wt %, about 5 wt % to about 60 wt %, about 5 wt % to about 55 wt %, about 5 wt % to about 50 wt %, about 5 wt % to about 45 wt %, about 5 wt % to about 40 wt %, about 5 wt % to about 35 wt %, about 5 wt % to about 30 wt %, about 5 wt % to about 25 wt %, about 5 wt % to about 20 wt %, about 5 wt % to about 15 wt %, about 5 wt % to about 10 wt %, about 10 wt % to about 100 wt %, about 10 wt % to about 95 wt %, about 10 wt % to about 90 wt %, about 10 wt % to about 85 wt %, about 10 wt % to about 80 wt %, about 10 wt % to about 75 wt %, about 10 wt % to about 70 wt %, about 10 wt % to about 65 wt %, about 10 wt % to about 60 wt %, about 10 wt % to about 55 wt %, about 10 wt % to about 50 wt %, about 10 wt % to about 45 wt %, about 10 wt % to about 40 wt %, about 10 wt % to about 35 wt %, about 10 wt % to about 30 wt %, about 10 wt % to about 25 wt %, about 10 wt % to about 20 wt %, about 10 wt % to about 15 wt %, or a value within one of these ranges. Specific examples may include about 0.1 wt %, about 0.25 wt %, about 0.5 wt %, about 0.75 wt %, about 1 wt %, about 3 wt %, about 5 wt %, about 10 wt %, about 15 wt %, about 20 wt %, about 25 wt %, about 30 wt %, about 35 wt %, about 40 wt %, about 45 wt %, about 50 wt %, about 60 wt %, about 70 wt %, about 80 wt %, about 90 wt %, about 100 wt %, about 110 wt %, about 120 wt %, about 130 wt %, about 140 wt %, about 150 wt %, about 160 wt %, about 170 wt %, about 180 wt %, about 190 wt %, about 200 wt %, about 210 wt %, about 220 wt %, about 230 wt %, about 240 wt %, about 250 wt %, about 260 wt %, about 270 wt %, about 280 wt %, about 290 wt %, about 300 wt %, or a range between any two of these values. In some embodiments, the therapeutically effective amount may be in an amount of about 3 wt % to about 90 wt %. The forgoing percentages are relative to a composition made from AB-101 with exemplary amounts of the marker compounds present in the latex as disclosed in Table 1. To illustrate, a therapeutically effective amount in the amount of 100 wt % of AB-101 will contain at least about 110 PPM of gallocatechin, while a therapeutically effective amount in the amount of 200 wt % of AB-101 will contain at least about 220 PPM of gallocatechin. The foregoing all representing weight percentages of the pharmaceutical composition.

In some embodiments, the therapeutically effective amount can vary according to, for example, the particular use for which the treatment is made, the manner of administration of the compound, the health and condition of the patient, and the judgment of the prescribing physician. The proportion or concentration of latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the composition contains at least the concentration of components of latex of Croton lechleri, preferably the concentration of components of filtered latex of Croton lechleri, preferably the concentration of components of filtered latex of Croton lechleri Müll.Arg of the reference standard, in a pharmaceutical composition can vary depending upon a number of factors including dosage, chemical characteristics (e.g., hydrophobicity), and the route of administration. For example, the compounds can be provided in an aqueous physiological buffer solution containing about 0.1 to about 10% w/v of the compound for parenteral administration. Some typical dose ranges for the compounds are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, composition of the excipient, and its route of administration. Effective doses can be extrapolated from dose-response curves derived from in vitro or animal model test systems.

The amount of composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications.

### Methods of Use

Cystic fibrosis is a progressive, genetic disease that causes persistent lung infections and limits the ability to breathe over time. In people with cystic fibrosis, mutations in the cystic fibrosis transmembrane conductance regulator (CFTR) gene cause the CFTR protein to become dysfunctional. When the protein is not working correctly, it's unable to help move chloride, a component of salt, to the cell surface. Without the chloride to attract water to the cell surface, the mucus in various organs becomes thick and sticky. In the lungs, the mucus clogs the airways and traps germs, like bacteria, leading to infections, inflammation, respiratory failure, and other complications. For this reason, minimizing contact with germs is a top concern for people with cystic fibrosis. Pseudomonas is a major cause of lung infections in people with cystic fibrosis. Pseudomonas is recognized as one of the most important pulmonary pathogens and the predominant cause of morbidity and mortality in cystic fibrosis. Some Pseudomonae have become resistant to commonly used antibiotics or drugs. Antibiotic or drug resistant bacteria are bacteria that are not controlled or killed by antibiotics or drugs. They are able to survive and even multiply in the presence of an antibiotic or drug. Most infection-causing bacteria can become resistant to at least some antibiotics or drugs. Bacteria that are resistant to many antibiotics are known as multi-resistant organisms (or MRO) or multi-drug resistant organisms (or MDRO). Treating cystic fibrosis and/or a bacterial infection in a cystic fibrosis patient via administration to the lungs can be a first line of defense to prevent the a bacterial infection from spreading and preventing detrimental and serious health effects including death.

The present invention relate to methods of treatment of cystic fibrosis and/or a bacterial infection in a cystic fibrosis patient in a subject comprising the administration of a therapeutically effective amount via inhalation of latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard, or a pharmaceutical composition containing latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the therapeutically effective amount contains at least the concentration of components of latex of Croton lechleri, preferably the concentration of components of filtered latex of Croton lechleri, preferably the concentration of components of filtered latex of Croton lechleri Müll.Arg of the reference standard, as disclosed herein. In some embodiments, the pharmaceutical composition may include a pharmaceutically acceptable excipient. As disclosed herein the latex of Croton lechleri, preferably filtered latex of Croton lechleri, preferably filtered latex of Croton lechleri Müll.Arg. where the components that have a negative impact on the delivery and/or treatment via inhalation have optionally been removed shall comprise one or more compounds selected from: gallocatechin, epigallocatechin, catechin, epicatechin, taspine, and dimethylcedrusin, and combinations thereof. Each of gallocatechin, epigallocatechin, catechin, epicatechin, taspine, and dimethylcedrusin may be present in the latex of Croton lechleri, preferably filtered latex of Croton lechleri, preferably filtered latex of Croton lechleri Müll.Arg. where the components that have a negative impact on the delivery and/or treatment via inhalation have optionally been removed is in the amounts found in Table 1a or paragraphs [0092]-[0097], or any combination of such amounts. In some embodiments latex of Croton lechleri, preferably filtered latex of Croton lechleri, preferably filtered latex of Croton lechleri Müll.Arg. has a PDI of embodiments disclosed herein.

Also provided herein is latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the concentration of components of latex of Croton lechleri, preferably the concentration of components of filtered latex of Croton lechleri, preferably the concentration of components of filtered latex of Croton lechleri Müll.Arg of the reference standard, as disclosed herein for use as a medicament. In some embodiments latex of Croton lechleri, preferably filtered latex of Croton lechleri, preferably filtered latex of Croton lechleri Müll.Arg. has a PDI of embodiments disclosed herein.

Also provided herein is latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the concentration of components of latex of Croton lechleri, preferably the concentration of components of filtered latex of Croton lechleri, preferably the concentration of components of filtered latex of Croton lechleri Müll.Arg of the reference standard, as disclosed herein for use as a medicament for the treatment of cystic fibrosis and/or a bacterial infection in a cystic fibrosis patient. In some embodiments latex of Croton lechleri, preferably filtered latex of Croton lechleri, preferably filtered latex of Croton lechleri Müll.Arg. has a PDI of embodiments disclosed herein.

Also provided is the use of latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the concentration of components of latex of Croton lechleri, preferably the concentration of components of filtered latex of Croton lechleri, preferably the concentration of components of filtered latex of Croton lechleri Müll.Arg of the reference standard, as disclosed herein as a medicament for the treatment of cystic fibrosis and/or a bacterial infection in a cystic fibrosis patient. In some embodiments latex of Croton lechleri, preferably filtered latex of Croton lechleri, preferably filtered latex of Croton lechleri Müll.Arg. has a PDI of embodiments disclosed herein.

Also provided is latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the concentration of components of latex of Croton lechleri, preferably the concentration of components of filtered latex of Croton lechleri, preferably the concentration of components of filtered latex of Croton lechleri Müll.Arg of the reference standard, as disclosed herein for use in the manufacture of a medicament for the treatment of cystic fibrosis and/or a bacterial infection in a cystic fibrosis patient. In some embodiments latex of Croton lechleri, preferably filtered latex of Croton lechleri, preferably filtered latex of Croton lechleri Müll.Arg. has a PDI of embodiments disclosed herein.

Also provided is the use of latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the concentration of components of latex of Croton lechleri, preferably the concentration of components of filtered latex of Croton lechleri, preferably the concentration of components of filtered latex of Croton lechleri Müll.Arg of the reference standard, as disclosed herein for the treatment of cystic fibrosis and/or a bacterial infection in a cystic fibrosis patient. In some embodiments latex of Croton lechleri, preferably filtered latex of Croton lechleri, preferably filtered latex of Croton lechleri Müll.Arg. has a PDI of embodiments disclosed herein.

Also provided herein is a method of treating cystic fibrosis and/or a bacterial infection in a cystic fibrosis patient comprising contacting the cystic fibrosis and/or a bacterial infection in a cystic fibrosis patient with latex of Croton lechleri, preferably filtered latex of Croton lechleri, preferably filtered latex of Croton lechleri Müll.Arg. where the components that have a negative impact on the delivery and/or treatment via inhalation have optionally been removed and wherein the concentration of components of latex of Croton lechleri, preferably the concentration of components of filtered latex of Croton lechleri, preferably the concentration of components of filtered latex of Croton lechleri Müll.Arg of the reference standard, as disclosed herein. In some embodiments latex of Croton lechleri, preferably filtered latex of Croton lechleri, preferably filtered latex of Croton lechleri Müll.Arg. has a PDI of embodiments disclosed herein.

Also provided herein is a method for achieving a therapeutic effect in a patient comprising the administration of a therapeutically effective amount via inhalation of latex of Croton lechleri, preferably filtered latex of Croton lechleri, preferably filtered latex of Croton lechleri Müll.Arg. components that have a negative impact on the delivery and/or treatment via inhalation have optionally been removed and wherein the concentration of components of latex of Croton lechleri, preferably the concentration of components of filtered latex of Croton lechleri, preferably the concentration of components of filtered latex of Croton lechleri Müll.Arg of the reference standard, as disclosed herein. In some embodiments latex of Croton lechleri, preferably filtered latex of Croton lechleri, preferably filtered latex of Croton lechleri Müll.Arg. has a PDI of embodiments disclosed herein.

In certain embodiments, the bacterial infection is selected from the group consisting of a Pseudomonas aeruginosa infection, a Multi drug resistant (MDR) Pseudomonas aeruginosa infection, a Staphylococcus aureus infection, a methicillin-resistant Staphylococcus aureus infection, and combinations thereof.

In certain embodiments, the bacterial infection is a Pseudomonas aeruginosa infection.

In certain embodiments, the bacterial infection is a MDR resistant Pseudomonas aeruginosa infection.

In certain embodiments, the bacterial infection is a Staphylococcus aureus infection.

In certain embodiments, the bacterial infection is a methicillin-resistant Staphylococcus aureus infection.

The pharmaceutical compositions may be administered via inhalation (including, for example, oral inhalation and/or nasal inhalation). The specific dose level for any particular patient will depend upon a variety of factors including the activity of the specific compound employed, the age, body weight, general health, sex, diet, time of administration, route of administration, rate of excretion, drug combination, the precise disorder being treated, and the severity of the indication or condition being treated. Also, the route of administration may vary depending on the condition and its severity.

Pharmaceutical compositions of the present invention may be administered once per day, twice per day, thrice per day, 4 times per day, 5 times per day, 6 times per day, 7 times per day, 8 times per day, 9 times per day, 10 times per day, or a range between of these values. In some embodiments, the pharmaceutical composition is administered twice per day. In some embodiments, the pharmaceutical composition is administered thrice per day. In some embodiments, the pharmaceutical composition is administered until the cystic fibrosis and/or a bacterial infection in a cystic fibrosis patient is resolved, gone, or treated.

Pharmaceutical compositions of the present invention may be administered continuously, every 15 minutes 30 min., 1 hour(s) (hr.), 1½ hr., 2 hr., 2½ hr., 3 hr., 4 hr., 6 hr., 8 hr., 12 hr., 24 hr., 36 hr., 48 hr., 3 days, 4 days, 5 days, 6, days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or a range between of these values. In some embodiments, the administration lasts 24 weeks. In particular embodiments, the administration lasts 2 weeks. In some embodiments, the administration lasts until the cystic fibrosis and/or a bacterial infection in a cystic fibrosis patient is resolved, gone, or treated.

Treatment of the cystic fibrosis and/or a bacterial infection in a cystic fibrosis patient will last 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or a range between of these values. In some embodiments, the treatment lasts 2 weeks. In some embodiments, the treatment lasts until the bacterial infection in a cystic fibrosis patient is resolved, gone, or treated.

Treatment of the bacterial infection in a cystic fibrosis patient may continue until complete resolution of the infection.

Treatment of the bacterial infection in a cystic fibrosis patient may continue at the discretion of the prescribing physician.

In certain embodiments, the pharmaceutical compositions of the present invention may be administered to the lungs via oral inhalation

In certain embodiments, the pharmaceutical compositions of the present invention may be administered to the lungs via nasal inhalation.

Administration to the lungs may be achieved by inhalation, and administration by inhalation herein may be oral and/or nasal. Examples of pharmaceutical devices for pulmonary delivery include metered dose inhalers (MDIs), dry powder inhalers (DPIs), and nebulizers.

Pressurized metered dose inhalers (pMDIs) are the most commonly used inhaler worldwide. The aerosol is created when a valve is opened (usually by pressing down on the propellant canister), allowing liquid propellant to spray out of a canister. Typically, a drug or therapeutic is contained in small particles (usually a few microns in diameter) suspended in the liquid propellant, but in some formulations the drug or therapeutic may be dissolved in the propellant. The propellant evaporates rapidly as the aerosol leaves the device, resulting in small drug or therapeutic particles that are inhaled. Propellants typically used in such pMDIs include but are not limited to hydrofluoroalkanes (HFAs). A surfactant may also be used, for example, to formulate the drug or therapeutic, with pMDIs. Other solvents or excipients may also be employed with pMDIs, such as ethanol, ascorbic acid, sodium metabisulfate, glycerin, chlorobutanol, and cetylpyridium chloride. Such pMDIs may further include add-on devices such as, for example, spacers, holding chambers and other modifications.

Nebulizers produce a mist of drug-containing liquid droplets for inhalation. They are usually classified into two types: ultrasonic nebulizers and jet nebulizers. A new type of nebulizer is also available, which does not require ultrasound or air pressure to function. Single breath atomizers have also been developed, which is used to deliver a drug in a single inhalation and may be preferred because of less contamination. Jet nebulizers are more common and use a source of pressurized air to blast a stream of air through a drug-containing water reservoir, producing droplets in a complex process involving a viscosity-induced surface instability that leads to nonlinear phenomena in which surface tension and droplet breakup on baffles play a role. Ultrasonic nebulizers produce droplets by mechanical vibration of a plate or mesh. In either type of nebulizer, the drug is usually contained in solution in the liquid in the nebulizer and so the droplets being produced contain drug in solution. However, for some formulations the pharmaceutical composition is contained in small particles suspended in the water, which are then contained as particles suspended inside the droplets being produced. Certain excipients are usually included in formulations suitable for nebulization, such as sodium chloride (e.g., to maintain isotonicity), mineral acids and bases (e.g., to maintain or adjust pH), nitrogen headspace sparging, benzalkonium chloride, calcium chloride, sodium citrate, disodium edtate, and polysorbate 80.

The third type of inhaler is the dry powder inhaler (DPI). In DPIs, the aerosol is usually a powder, contained within the device until it is inhaled. The therapeutic or drug is manufactured in powder form as small powder particles (usually a few millionths of a meter, or micrometers, in diameter). In many DPIs, the drug or therapeutic is mixed with much larger sugar particles (e.g., lactose monohydrate), that are typically 50-100 micrometers in diameter. The increased aerodynamic forces on the lactose/drug agglomerates improve entrainment of the drug particles upon inhalation, in addition to allowing easier filling of small individual powder doses. Upon inhalation, the powder is broken up into its constituent particles with the aid of turbulence and/or mechanical devices such as screens or spinning surfaces on which particle agglomerates impact, releasing the small, individual drug powder particles into the air to be inhaled into the lung. The sugar particles are usually intended to be left behind in the device and/or in the mouth-throat.

Additional methods of methods of administration to the lungs are known to those of skill in the art and may be found in, for example, U.S. Pat. No. 8,003,081; U.S. U.S. Pat. Nos. 8,168,598; 9,345,663; 9,856,283; U.S. 2012/0076859; U.S. 2018/0360864; WO2010043981; WO2011038901, which are hereby incorporated by reference.

In some embodiments, the pharmaceutical composition has a minimum inhibitory concentration (MIC) of at least about 0.01%, at least about 0.05%, at least about 0.1%, at least about 0.25%, at least about 0.5%, at least about 0.75%, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%.

### Example 1—Evaluation of AB-101 Antibacterial Activity

An Agar Well Diffusion Assay pilot evaluation of AB-101 BRM was conducted to seek an initial signal of the activity of AB-101 BRM against various bacteria. AB-101 BRM was tested against the following bacterial species:

| | |
|---|---|
| Pseudomonas aeruginosa (ATCC 27853) (PAER) | Gram (−) |
| Staphylococcus aureus (ATCC 52923) (SAUR) | Gram (+) |
| Methicillin-resistant Staphylococcus aureus | Gram (+) |
| (ATCC 49476) (MRSA) | |

Isolates were subcultured the day before the Agar Well Diffusion Assay was performed. On the day of the assay, a bacterial suspension (0.08-0.12 MacFarland units) of each bacteria isolate was made in Trypticase Soy Broth (TSB), and these suspensions were used to plate a lawn of bacterial organisms on Mueller-Hinton agar or Mueller-Hinton sheep's blood agar. A control antibiotic Kirby-Bauer (K-B) disk plus K-B disks containing 25 uL of AB-101 dilutions were placed on the plates, and the plates were incubated overnight. The next day, the width of the zone of growth inhibition surrounding each K-B disk was recorded (in mm). The larger the number, the greater the zone of inhibition, and the greater the inhibition of growth of the target organisms.

AB-101 was used undiluted (AB⁰) and in 10-fold dilutions (AB⁻¹, AB⁻², AB⁻³, AB⁻⁴) made in dimethylsulfoxide (DMSO). Control experiments showed that AB-101 BRM precipitated when mixed into either TSB or Hanks Balanced Salt Solution with Ca⁺⁺ and Mg⁺⁺, but was apparently soluble in DMSO, ethanol or methanol. However, when initially diluted into DMSO, EtOH or MeOH at 1:10 and subsequently diluted into TSB, AB-101 continued to precipitate. Therefore, all dilutions were made into DMSO and a K-B disk containing 25 uL DMSO was included in all experiments as a vehicle control. Table 3 provides a summary of the results. Table 4 (AMP=Ampicillin; GM=Gentamicin) provides the individual results of the Zone of Inhibition Assay for AB-101. As shown in Table 3 AB-101 is effective against both Gram (+) and Gram (−) bacteria.

TABLE 3

| | | Bacteria | AB-101 |
|---|---|---|---|
| Bacteria | Abbreviation | Type | Activity |
| Pseudomonas aeruginosa | (PAER) | Gram (−) | Yes |
| Staphylococcus aureus | (SAUR) | Gram (+) | Yes |
| Methicillin-resistant | (MRSA) | Gram (+) | Yes |
| Staphylococcus aureus | | | |

TABLE 4

| | Antibiotic | Zone of Inhibition (MM) | | | | | |
|---|---|---|---|---|---|---|---|
| | Controls | DMSO | AB⁰ | AB⁻¹ | AB⁻² | AB⁻³ | AB⁻⁴ |
| PAER | Gram (−) | | | | | | |
| Jun. 3, 2005 | GM 7 | 0 | 4 | 1 | 1 | <1 | <1 |
| Jun. 6, 2005 | GM 10 | <1 | 1 | 1 | <1 | <1 | <1 |
| Jun. 7, 2005 | GM 8 | <1 | <1 | <1 | <1 | <1 | <1 |
| SAUR | Gram (+) | | | | | | |
| Jun. 2, 2005 | GM 6 | 0 | 3 | 2 | <1 | <1 | <1 |
| Jun. 3, 2005 | GM 7 | 0 | 3 | 2 | <1 | <1 | <1 |
| Jun. 6, 2005 | GM 8 | <1 | 3 | 3 | 1 | <1 | <1 |
| Jun. 7, 2005 | GM 8 | 0 | 4 | 4 | <1 | <1 | <1 |
| MRSA | Gram (+) | | | | | | |
| Jun. 3, 2005 | GM 1 | 0 | 5 | 3 | <1 | <1 | <1 |
| Jun. 6, 2005 | GM <1 | <1 | 4 | 4 | <1 | <1 | <1 |
| Jun. 7, 2005 | GM 1 | 0 | 5 | 4 | <1 | <1 | <1 |

When evaluating the preliminary K-B data, there was a dilution effect on inhibition which validated this testing. The zones of inhibition are 4-5 mm at the highest concentration, which is indicative of antibacterial activity especially since no standard for what constitutes activity exists for the compounds in AB-101. When assessing antibiotic effects in a K-B type test, the minimum zone of inhibition for considering a positive test varies depending on the antibiotic tested with anything more than 0 mm considered positive in some cases or as much as 10 mm or more needed for others. The laboratory concluded that with AB-101, any inhibition should be considered a preliminary positive test and confirmed with more rigorous testing, and that AB-101's actual antibacterial activity may be greater than what was seen in these experiments. AB-101 was insoluble in aqueous buffer which likely limited its diffusion into agar further indicating anything more than 0 mm for AB-101 should be considered positive.

As shown in Table 4, AB-101 had activity against the following: Staphylococcus aureus, Methicillin-resistant Staphylococcus aureus, and Pseudomonas aeruginosa.

### Example 2—Antibacterial Testing of AB-101 Against Staphylococcus aureus

AB-101 was tested against an extensive variety of bacterial strains, including Mupirocin-resistant MRSA, Mupirocin-resistant MSSA, Mupirocin-susceptible MSSA, Mupirocin-susceptible MRSA, and other skin-related pathogens.

The initial antimicrobial testing of AB-101 is described below. The efficacy of AB-101 was assessed against ATCC 29213, an MSSA strain and ATCC 33591, an MRSA strain, using a variety of different microbiological assays in an attempt to identify the best approach at determining AB-101 sensitivity.

The first assay performed was the agar diffusion assay. A lawn of bacteria was painted on an agar plate, and the surface was then gently incised with a 6 mm biopsy punch. A 10 μL droplet of sample was then applied inside the ring incised by the biopsy punch. Three lots of AB-101 were assessed and test conditions were conducted in duplicates. After the agar plates were incubated, the diameter of the Zone of Inhibition (ZOI) was measured with a ruler and the average diameter was calculated; for samples where the ZOI was not circular, the diameter of the narrowest portion of the ZOI was measured. Table 5 provides the average zone of inhibition of AB-101 resin in the agar diffusion assay.

TABLE 5

| | Average zone of inhibition (mm) of AB-101 resin | | |
|---|---|---|---|
| | AB-101 | AB-101 | AB-101 |
| Strain | Lot 00 | Lot 01 | Lot 02 |
| ATCC 29213 (MSSA) | 8.0 | 8.5 | 7.5 |
| ATCC 33591 (MRSA) | 8.5 | 9.0 | 9.0 |

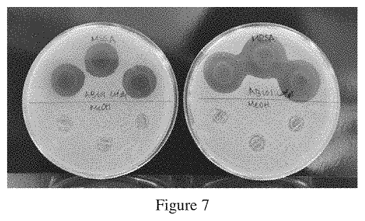

The ZOI for AB-101 was around 7.5 to 9.0 mm and were similar between MRSA and MSSA. The surface of AB-101 droplets tend to form a film after exposure to air, resulting in higher surface tension and limiting its ability to flow and spread across the surface of the agar. Extractions of raw, botanical material are often performed prior to antimicrobial testing. A methanol extraction of AB-101 by lyophilizing the resin to remove its water content, then resuspending the dried powder to 250 mg/mL with pure methanol. Each lot of methanol extracted AB-101 was then tested in an agar diffusion assay in triplicates, and methanol-only controls were included for each strain to assess the antimicrobial contribution of the solvent. The average ZOI of the methanol extracted AB-101 is summarized in Table 6. A representative image of the ZOI of methanol extracted AB-101 against MSSA (on the left) and MRSA (on the right) is shown in FIG. 7, with the methanol extract on the top of the plates and the methanol controls on the bottom of the plates.

TABLE 6

| | Average zone of inhibition (mm) | | | |
|---|---|---|---|---|
| | of methanol extracted AB-101 | | | |
| | AB-101 | AB-101 | AB-101 | Methanol |
| Strain | Lot 00 | Lot 01 | Lot 02 | control |
| ATCC 29213 (MSSA) | 17.3 | 18.3 | 16.7 | 9.1 |
| ATCC 33591 (MRSA) | 20.0 | 23.7 | 20.7 | 8.8 |

The average ZOI of methanol extracted AB-101 were between 16.7 to 23.7 mm, roughly double that of the methanol-only control (Table 7). In addition, the ZOI of the methanol control as seen in Table 7 is hazy, indicating the methanol evaporated quickly after being dispensed on the agar and did not greatly inhibit bacterial growth. This contrasts with methanol extracted AB-101, which exhibit very clear ZOI, indicative of a strong antimicrobial effect from AB-101.

In addition to agar diffusion assay, the antimicrobial activity of AB-101 was assessed in broth microdilution assay. Both AB-101 and methanol extracted AB-101 were tested by 2-fold serial dilutions into the bacteriological media, Cation-Adjusted Mueller-Hinton Broth (CAMHB). An equal volume of bacteria inoculum was then added to the serially diluted samples in a 96 well plate to generate the final test concentrations in Table 7. A methanol-only control was also included to determine the contribution of the solvent for the methanol extracted AB-101. All conditions were tested in duplicates and the 96 well plates were incubated overnight for approximately 20 hours at 37° C.

TABLE 7
Concentration of AB-101 and methanol extracted AB-101 tested in broth microdilution assay

| Well # | 1 | 2 | 3 | 4 | 5 | 6 | 7 | 8 | 9 | 10 | 11 | 12 |
|---|---|---|---|---|---|---|---|---|---|---|---|---|
| AB-101 (%) | 50 | 25 | 12.5 | 6.25 | 3.125 | 1.563 | 0.781 | 0.391 | 0.195 | 0.098 | 0.049 | 0 |
| Methanol AB- | 125 | 62.5 | 31.25 | 15.625 | 7.8125 | 3.906 | 1.953 | 0.977 | 0.488 | 0.244 | 0.122 | 0 |
| 101 (mg/mL) | | | | | | | | | | | | |
| Methanol | 50 | 25 | 12.5 | 6.25 | 3.125 | 1.563 | 0.781 | 0.391 | 0.195 | 0.098 | 0.049 | 0 |
| control (%) | | | | | | | | | | | | |

Both AB-101 and the methanol extracted AB-101 formed cloudy, brownish precipitates in the presence of the water-based CAMHB media. To assess the viability of bacteria in the cloudy suspension, 10 μL from each assay condition was drop-platted on agar and incubated to determine the Minimum Bactericidal Concentration (MBC), which is defined as the lowest concentration of a test solution from which no colonies are recovered after incubation. The MBC values were determined below in Table 8. The MBC value of the methanol extracted AB-101 is considerable lower than the methanol-only control, a result that is similar to the agar diffusion assay performed with this form of AB-101 (see Table 8), and reinforces the observation that methanol extracted AB-101 has potent antimicrobial efficacy against Staphylococcus aureus, which includes MRSA, Mupirocin-resistant MRSA and MSSA that is independent from the solvent.

TABLE 8
MBC values of AB-101 and methanol extracted
AB-101 tested in broth microdilution assay

| | AB-101 | | Methanol Extracted AB-101 | | Methanol control | |
|---|---|---|---|---|---|---|
| Strain | MBC (%) | | MBC (mg/mL) | | MBC (%) | |
| MSSA | 50 | 50 | 0.977 | 0.977 | 50 | 50 |
| (ATCC | | | | | | |
| 29213) | | | | | | |
| MRSA | 50 | 50 | 0.977 | 0.977 | 50 | 50 |
| (ATCC | | | | | | |
| 33591) | | | | | | |

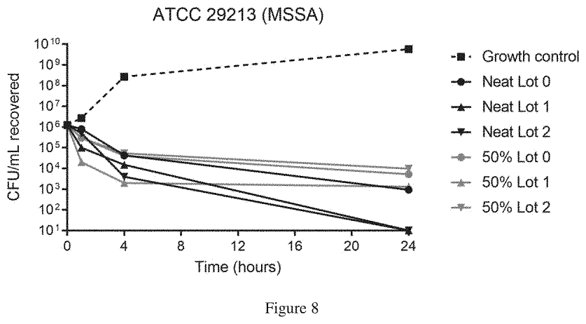

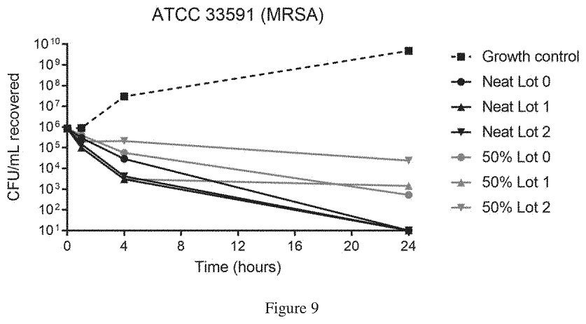

While the precipitation of AB-101, both in BRM and methanol extracted form, possesses a significant technical challenge for rapid antimicrobial screening, we noticed that the MBC value for AB-101 BRM is 50%. This indicates AB-101 is bactericidal in the range of 100% to 50% within 20 hours of contact with S. aureus. To determine how quickly and to what extent AB-101 exerts its bactericidal effect, a time-kill kinetics assay was performed against MRSA and MSSA. Undiluted AB-101 BRM or AB-101 diluted to 50% (v/v) with CAMHB were inoculated with MSSA or MRSA. Bacteria density was enumerated on agar plates at 1, 4, and 24 hours after inoculation and graphed relative to the time 0 inoculum density. The CFU/mL recovered and graphed data for MSSA (Table 9 and FIG. 8) and MRSA (Table 10 and FIG. 9) are shown below. In Table 9 CFU/mL values for Undiluted AB-101 BRM Lot 001 at T24 and Undiluted AB-101 BRM Lot 002 at T24 and in Table 10 CFU/mL values for Undiluted AB-101 BRM Lot 00 at T24, Undiluted AB-101 BRM Lot 001 at T24 and Undiluted AB-101 BRM Lot 002 at T24 indicate samples from which no colonies were recovered and represents the limit of detection of the assay (ie, 10 CFU/mL).

TABLE 9
MSSA CFU/mL recovered in time-kill assay

| | CFU/mL recovered | | | |
|---|---|---|---|---|
| ATCC 29213 (MSSA) | T0 | T1 | T4 | T24 |
| Growth control | 1.2E+06 | 2.7E+06 | 2.7E+08 | 5.8E+09 |
| Undiluted AB-101 | | 8.0E+05 | 4.3E+04 | 9.3E+02 |
| Lot 00 | | | | |
| Undiluted AB-101 | | 1.0E+05 | 1.5E+04 | 1.0E+01 |
| Lot 01 | | | | |
| Undiluted AB-101 | | 5.0E+05 | 4.0E+03 | 1.0E+01 |
| Lot 02 | | | | |
| Diluted AB-101 | | 3.0E+05 | 4.1E+04 | 5.2E+03 |
| 50% Lot 00 | | | | |
| Diluted AB-101 | | 2.0E+04 | 2.0E+03 | 1.3E+03 |
| 50% Lot 01 | | | | |
| Diluted AB-101 | | 3.0E+05 | 5.4E+04 | 9.7E+03 |
| 50% Lot 02 | | | | |

TABLE 10
MRSA CFU/mL recovered in time-kill assay

| | CFU/mL recovered | | | |
|---|---|---|---|---|
| ATCC 33591 (MRSA) | T0 | T1 | T4 | T24 |
| Growth control | 8.5E+05 | 9.0E+05 | 3.0E+07 | 4.8E+09 |
| Neat Lot 00 | | 3.0E+05 | 2.9E+04 | 1.0E+01 |
| Neat Lot 01 | | 1.0E+05 | 3.0E+03 | 1.0E+01 |
| Neat Lot 02 | | 1.5E+05 | 4.2E+03 | 1.0E+01 |
| 50% Lot 00 | | 4.0E+05 | 5.7E+04 | 5.3E+02 |
| 50% Lot 01 | | 1.0E+05 | 3.1E+03 | 1.4E+03 |
| 50% Lot 02 | | 2.0E+05 | 2.1E+05 | 2.4E+04 |

Compared to the growth control (which contained no AB-101), both the undiluted AB-101 BRM and the 50% diluted AB-101 BRM reduced the bacterial density by 2 to 3 Log 10 CFUs 4 hours post inoculation. By 24 hours, the MSSA and MRSA densities were reduced by ≥7 Log 10 CFUs compared to growth control, and nearly all undiluted AB-101 test samples reduced the bacterial density to a level that is below the limit of detection for this assay. The time-kill assay therefore further confirmed the bactericidal effect of AB-101 against S. aureus.

### Example 3

To demonstrate the effectiveness of AB-101 and how changes within different type of latex extract can change performance and well as showing that not all extracts, even in the same plant species, yields the same pharmaceutical grade performance, invitro assay of MSSA and MRSA were conducted. Measures included the minimum inhibitory concentration (MIC) which is the lowest concentration of AB-101 that prevents visible growth of the bacterium or pathogen, and minimum bactericidal concentration (MBC) which is the lowest concentration of an antibacterial agent required to kill a bacterium.

The comparison of AB-101 Lot 01 and 2 for MIC demonstrates a high effectiveness against MSSA and MRSA with particular emphasis on the Mupirocin resistant strain of MRSA. Mupirocin is a leading topical treatment for MRSA. Shown for the first time is the effectiveness of AB-101 against these pathogens and importantly an improvement over the leading current pharmaceutical treatment. Results are shown in Table 11.

TABLE 11

| | | MIC (% vol./vol.) | | | | MIC (μg/mL) | |
|---|---|---|---|---|---|---|---|
| Strain ID | Characteristic | AB-101 Lot 01 | | AB-101 Lot 02 | | Methicillin | Mupirocin |
| CDC 218 | MupirocinR, MRSA | 12.5 | 12.5 | 12.5 | 12.5 | >64 | >256 |
| CDC 224 | MupirocinR, MRSA | 12.5 | 12.5 | 12.5 | 12.5 | >64 | >256 |
| CDC 228 | MupirocinR, MRSA | 3.13 | 3.13 | 3.13 | 3.13 | 64 | >256 |
| 1674606 | MupirocinR, MSSA | 50 | 50 | 50 | 50 | 2-8 | >256 |
| 1674607 | MupirocinR, MRSA | 12.5 | 12.5 | 12.5 | 12.5 | >64 | >256 |
| 1674608 | MupirocinR, MSSA | 12.5 | 12.5 | 25 | 50 | 2-4 | >256 |
| 1674611 | MupirocinR, MRSA | 6.25 | 6.25 | 12.5 | 12.5 | 64 | >256 |
| CDC 480 | MupirocinS, MRSA | 12.5 | 25 | 12.5 | 25 | 32-64 | ≤0.25 |
| CDC 481 | MupirocinS, MRSA | 12.5 | 12.5 | 12.5 | 12.5 | 16-32 | ≤0.25 |
| CDC 482 | MupirocinS, MRSA | 12.5 | 12.5 | 12.5 | 12.5 | 8-32 | ≤0.25 |
| CDC 483 | MupirocinS, MRSA | 12.5 | 12.5 | 12.5 | 12.5 | 64 | ≤0.25 |
| CDC 220 | MupirocinS, MRSA | 12.5 | 12.5 | 6.25 | 12.5 | 64->64 | ≤0.25 |
| CDC 227 | MupirocinS, MRSA | 6.25 | 6.25 | 12.5 | 12.5 | 64 | ≤0.25-0.5 |
| CDC 461 | MupirocinS, MSSA | 12.5 | 12.5 | 12.5 | 12.5 | 4-8 | ≤0.25-1 |
| CDC 462 | MupirocinS, MSSA | 6.25 | 12.5 | 6.25 | 12.5 | 8 | ≤0.25 |
| CDC 463 | MupirocinS, MSSA | 6.25 | 6.25 | 12.5 | 12.5 | 8-16 | ≤0.25-0.5 |
| CDC 464 | MupirocinS, MSSA | 12.5 | 12.5 | 12.5 | 12.5 | 2 | ≤0.25 |
| CDC 484 | MupirocinS, MSSA | 12.5 | 12.5 | 12.5 | 12.5 | 2 | ≤0.25 |
| CDC 485 | MupirocinS, MSSA | 50 | 50 | 50 | 50 | 2-4 | ≤0.25 |
| ATCC 29213 | QC control | 12.5 | 50-12.5 | 12.5 | 12.5 | 1-2 | ≤0.25-0.5 |

Table 12 shows the comparison of AB-101 Lots 01 and 02 for MBC demonstrates a high effectiveness against MSSA and MRSA with particular emphasis on the mupirocin resistant strain of MRSA. Once again Mupirocin is a leading topical treatment for MRSA. Shown for the first time is the effectiveness of AB-101 against these pathogens and importantly an improvement over the leading current pharmaceutical treatment.

TABLE 12

| | | MBC (% vol./vol.) | | | |
|---|---|---|---|---|---|
| | | AB-101 | | AB-101 | |
| Strain ID | Characteristic | Lot 1 | | Lot 2 | |
| CDC 218 | MupirocinR, MRSA | 50 | 50 | 50 | 50 |
| CDC 224 | MupirocinR, MRSA | 50 | 50 | 50 | 50 |
| CDC 228 | MupirocinR, MRSA | 6.25 | 6.25 | 6.25 | 6.25 |
| 1674606 | MupirocinR, MSSA | >50 | >50 | >50 | >50 |
| 1674607 | MupirocinR, MRSA | 12.5 | 12.5 | 25 | 25 |
| 1674608 | MupirocinR, MSSA | 50 | 50 | 50 | >50 |
| 1674611 | MupirocinR, MRSA | 12.5 | 12.5 | 50 | 50 |
| CDC 480 | MupirocinS, MRSA | 50 | 50 | 50 | 50 |
| CDC 481 | MupirocinS, MRSA | 50 | 50 | 50 | 50 |
| CDC 482 | MupirocinS, MRSA | 50 | 50 | 50 | 50 |
| CDC 483 | MupirocinS, MRSA | 50 | 50 | 50 | 50 |
| CDC 220 | MupirocinS, MRSA | 25 | 25 | 25 | 25 |
| CDC 227 | MupirocinS, MRSA | 12.5 | 12.5 | 25 | 25 |
| CDC 461 | MupirocinS, MSSA | 50 | 50 | 50 | 50 |
| CDC 462 | MupirocinS, MSSA | 12.5 | 50 | 50 | 50 |
| CDC 463 | MupirocinS, MSSA | 12.5 | 12.5 | 12.5 | 12.5 |
| CDC 464 | MupirocinS, MSSA | >50 | 50 | 50 | 50 |
| CDC 484 | MupirocinS, MSSA | 50 | 50 | 50 | 50 |
| CDC 485 | MupirocinS, MSSA | 50 | 50 | 50 | 50 |
| ATCC | QC control | 50-12.5 | 50-12.5 | 50 | 50 |
| 29213 | | | | | |

Table 13 compares AB-101 lot X and Lot 00 for MIC because these lots have been shown elsewhere to have different composition. Lot X and Lot 00 are latex extracts of Croton lechleri Müll.Arg., the same species, grown in similar locations. Lot X demonstrates a significantly higher effectiveness against MSSA and MRSA. This data shows for the first time that not all latex extracts of Croton lechleri Müll.Arg. provide the same performance, even when the extracts are from the same species grown in similar locations.

TABLE 13

| | | MIC (% vol./vol.) | | | | MIC (pg/mL) | |
|---|---|---|---|---|---|---|---|
| Strain ID | Characteristic | AB-101 Lot 00 | | AB-101 Lot X | | Methicillin | Mupirocin |
| CDC 218 | MupirocinR, MRSA | 12.5 | 12.5 | 0.78 | 0.78 | >64 | >256 |
| CDC 224 | MupirocinR, MRSA | 12.5 | 12.5 | 0.78 | 0.78 | >64 | >256 |
| CDC 228 | MupirocinR, MRSA | 3.13 | 3.13 | 0.39 | 0.39 | 64 | >256 |
| 1674606 | MupirocinR, MSSA | >50 | >50 | 1.56 | 1.56 | 2-8 | >256 |
| 1674607 | MupirocinR, MRSA | 12.5 | 12.5 | 0.78 | 0.78 | >64 | >256 |
| 1674608 | MupirocinR, MSSA | 25 | 25 | 1.56 | 1.56 | 2-4 | >256 |
| 1674611 | MupirocinR, MRSA | 25 | 25 | 0.78 | 0.78 | 64 | >256 |
| CDC 480 | MupirocinS, MRSA | 12.5 | 12.5 | 1.56 | 1.56 | 32-64 | ≤0.25 |
| CDC 481 | MupirocinS, MRSA | 12.5 | 12.5 | 1.56 | 1.56 | 16-32 | ≤0.25 |
| CDC 482 | MupirocinS, MRSA | 12.5 | 12.5 | 0.78 | 0.78 | 8-32 | ≤0.25 |
| CDC 483 | MupirocinS, MRSA | 12.5 | 12.5 | 0.78 | 0.78 | 64 | ≤0.25 |
| CDC 220 | MupirocinS, MRSA | 12.5 | 12.5 | 0.78 | 0.78 | 64->64 | ≤0.25 |
| CDC 227 | MupirocinS, MRSA | 12.5 | 12.5 | 0.78 | 0.78 | 64 | ≤0.25-0.5 |
| CDC 461 | MupirocinS, MSSA | 50 | 50 | 0.78 | 0.78 | 4-8 | ≤0.25-1 |
| CDC 462 | MupirocinS, MSSA | 12.5 | 12.5 | 0.78 | 0.78 | 8 | ≤0.25 |
| CDC 463 | MupirocinS, MSSA | 12.5 | 12.5 | 1.56 | 0.78 | 8-16 | ≤0.25-0.5 |
| CDC 464 | MupirocinS, MSSA | >50 | >50 | 1.56 | 1.56 | 2 | ≤0.25 |
| CDC 484 | MupirocinS, MSSA | >50 | 25 | 0.78 | 1.56 | 2 | ≤0.25 |
| CDC 485 | MupirocinS, MSSA | >50 | >50 | 0.78 | 0.78 | 2-4 | ≤0.25 |
| ATCC 29213 | QC control | 12.5 | 12.5 | 1.56 | 1.56 | 1-2 | ≤0.25-0.5 |

Table 14 compares AB-101 lot X and Lot 00 for MBC because these lots have been shown elsewhere to have different composition. Lot X and Lot 00 are latex extracts of Croton lechleri Müll.Arg., the same species, grown in similar locations. Lot X demonstrates a significantly higher effectiveness against MSSA and MRSA. This data shows for the first time that not all latex extracts of Croton lechleri Müll.Arg. provide the same performance, even when the extracts are from the same species grown in similar locations.

TABLE 14

| | | MBC (% vol./vol.) | | | |
|---|---|---|---|---|---|
| | | AB-101 | | AB-101 | |
| Strain ID | Characteristic | Lot 00 | | Lot X | |
| CDC 218 | MupirocinR, MRSA | >50 | 50 | 0.78 | 0.78 |
| CDC 224 | MupirocinR, MRSA | >50 | >50 | 0.78 | 0.78 |
| CDC 228 | MupirocinR, MRSA | 12.5 | 12.5 | 0.39 | 0.78 |
| 1674606 | MupirocinR, MSSA | >50 | >50 | 1.56 | 1.56 |
| 1674607 | MupirocinR, MRSA | 25 | 25 | >50 | >50 |
| 1674608 | MupirocinR, MSSA | >50 | >50 | 1.56 | 1.56 |
| 1674611 | MupirocinR, MRSA | 50 | >50 | 0.78 | 0.78 |
| CDC 480 | MupirocinS, MRSA | >50 | >50 | 1.56 | 3.12 |
| CDC 481 | MupirocinS, MRSA | >50 | >50 | 1.56 | 1.56 |
| CDC 482 | MupirocinS, MRSA | >50 | >50 | 0.78 | 0.78 |
| CDC 483 | MupirocinS, MRSA | >50 | >50 | 0.78 | 0.78 |
| CDC 220 | MupirocinS, MRSA | 50 | 50 | 0.78 | 1.56 |
| CDC 227 | MupirocinS, MRSA | 25 | 25 | 1.56 | 0.78 |
| CDC 461 | MupirocinS, MSSA | >50 | >50 | 0.78 | 0.78 |
| CDC 462 | MupirocinS, MSSA | 50 | 50 | 0.78 | 0.78 |
| CDC 463 | MupirocinS, MSSA | 12.5 | 12.5 | 6.25 | 6.25 |
| CDC 464 | MupirocinS, MSSA | >50 | >50 | 1.56 | 1.56 |
| CDC 484 | MupirocinS, MSSA | >50 | >50 | 1.56 | 1.56 |
| CDC 485 | MupirocinS, MSSA | >50 | >50 | >50 | >50 |
| ATCC | QC control | ≥50 | ≥50 | 6.25 | 3.13 |
| 29213 | | | | | |

To demonstrate the effectiveness of AB-101 across other pathogens and in particular across gram negative (−) pathogens, Lot 01 and 02 were tested against Pseudomonas aeruginosa. All 20 of the Pseudomonas aeruginosa tested are resistant to multiple antibiotics, thereby they all fit the definition of being Multi-Drug Resistant (MDR). Of the 20, 5 are known to be Verona integron-encoded metallo-beta-lactamase (VIM)-producing Pseudomonas aeruginosa which are drug resistant strains of Pseudomonas aeruginosa, another 5 are know to be Klebsiella pneumoniae carbapenemase (KPC)producing Pseudomonas aeruginosa strains which are drug resistant strains of Pseudomonas aeruginosa, and 4 are known to be IMP-Type Metallo-β-Lactamase (IMP))-producing Pseudomonas aeruginosa which are drug resistant strains of Pseudomonas aeruginosa. The 6 remaining strains are known to be antibiotic resistant and are listed simply as MDR strains. Measures included the minimum inhibitory concentration (MIC) which is the lowest concentration of AB-101 that prevents visible growth of the bacterium or pathogen, and minimum bactericidal concentration (MBC) which is the lowest concentration of an antibacterial agent required to kill a bacterium.

Table 15 shows the comparison of AB-101 Lot 01 and 02 for MIC demonstrates a high effectiveness against Pseudomonas aeruginosa. Shown for the first time is the effectiveness of AB-101 against these pathogens and importantly an improvement over the leading current pharmaceutical treatment.

TABLE 15

| Pseudomonas | Pseudomonas | | | | | | | | |
|---|---|---|---|---|---|---|---|---|---|
| aeruginosa | aeruginosa | MIC (% AB-101)^{a} | | | | MIC (μg/mL)^{b} | | | |
| Strain ID | Characteristic | Lot 1 | | Lot 2 | | Imipenem | | Ciprofloxacin | |
| CDC 0230 | VIM | 25 | 25 | 25 | 25 | >64 | >64 | >64 | >64 |
| CDC 0239 | VIM | 12.5 | 12.5 | 12.5 | 12.5 | >64 | >64 | 32 | 32 |
| CDC 0254 | VIM | 25 | 25 | 25 | 25 | >64 | >64 | 32 | 32 |
| CDC 0255 | VIM | 25 | 25 | 25 | 25 | >64 | >64 | 32 | 32 |
| CDC 0509 | VIM | 25 | 25 | 25 | 25 | >64 | >64 | 32 | 32 |
| CDC 0231 | KPC | 25 | 25 | 25 | 25 | >64 | >64 | >64 | >64 |
| CDC 0356 | KPC | 25 | 25 | 25 | 25 | >64 | >64 | 0.125 | 0.125 |
| CDC 0441 | KPC | 25 | 25 | 25 | 25 | >64 | >64 | 2 | 2 |
| CDC 0516 | KPC | 25 | 25 | 25 | 25 | >64 | >64 | 0.125 | 0.125 |
| CDC 0518 | KPC | 12.5 | 12.5 | 12.5 | 12.5 | >64 | >64 | >64 | >64 |
| CDC 0092 | IMP | 12.5 | 12.5 | 12.5 | 50 | >64 | >64 | 32 | 32 |
| CDC 0103 | IMP | 12.5 | 12.5 | 12.5 | 12.5 | >64 | >64 | >32 | >32 |
| CDC 0439 | IMP | 12.5 | 12.5 | 12.5 | 12.5 | >64 | >64 | 32 | 32 |
| CDC 0241 | IMP | 12.5 | 12.5 | 12.5 | 12.5 | >64 | >64 | 16 | 16 |
| CDC 0508 | MDR | 12.5 | 12.5 | 12.5 | 12.5 | 16 | 16 | 2 | 2 |
| CDC 0353 | MDR | 25 | 25 | 12.5 | 12.5 | 16 | 16 | 16 | 16 |
| CDC 0357 | MDR | 25 | 25 | 25 | 25 | 16 | 16 | 32 | 32 |
| CDC 0246 | MDR | 12.5 | 12.5 | 12.5 | 12.5 | >64 | >64 | 32 | 32 |
| CDC 0250 | MDR | 25 | 12.5 | 12.5 | 25 | >64 | >64 | 32 | 32 |
| CDC 0064 | MDR | 25 | 25 | 25 | 25 | >64 | >64 | 16 | 16 |
| ATCC 27853 | QC strain | 25 | 25 | 25 | 25 | 2 | 2 | 0.25 | 0.25 |

Table 16 shows the comparison of Iminipenem and Cripofloxacin for MIC against quality control strain ATCC 27853.

TABLE 16

| | |
|---|---|
| Pseudomonas | |
| aeruginosa | CLSI QC Range MIC (μg/ml) |

| Strain | Imipenem | Ciprofloxacin |
|---|---|---|
| ATCC 27853 | 1-4 | 0.125-1 |

Table 17 shows the comparison of AB-101 Lots 01 and 02 for MBC demonstrates a high effectiveness against Pseudomonas aeruginosa. Shown for the first time is the effectiveness of AB-101 against these pathogens specifically for the multi-drug resistant pathogens.

TABLE 17

| Pseudomonas | | | | | | |
|---|---|---|---|---|---|---|
| aeruginosa | Pseudomonas | Pseudomonas | | | | |
| screen | aeruginosa | aeruginosa | MBC (% AB-101) | | | |
| number | ID | Characteristic | Lot 01 | | Lot 02 | |
| 1 | CDC 0230 | VIM | 25 | 25 | 25 | 25 |
| 2 | CDC 0239 | VIM | 25 | 25 | 25 | 25 |
| 3 | CDC 0254 | VIM | 25 | 25 | 25 | 25 |
| 4 | CDC 0255 | VIM | 25 | 25 | 25 | 25 |
| 5 | CDC 0509 | VIM | 25 | 25 | 25 | 25 |
| 6 | CDC 0231 | KPC | 25 | 25 | 25 | 25 |
| 7 | CDC 0356 | KPC | 25 | 25 | 25 | 25 |
| 8 | CDC 0441 | KPC | 25 | 25 | 25 | 25 |
| 9 | CDC 0516 | KPC | 25 | 25 | 25 | 25 |
| 10 | CDC 0518 | KPC | 25 | 25 | 25 | 25 |
| 11 | CDC 0092 | IMP | 25 | 25 | 25 | 50 |
| 12 | CDC 0103 | IMP | 25 | 25 | 25 | 25 |
| 13 | CDC 0439 | IMP | 25 | 25 | 25 | 25 |
| 14 | CDC 0241 | IMP | 25 | 12.5 | 25 | 25 |
| 15 | CDC 0508 | MDR | 50 | 50 | 25 | 25 |
| 16 | CDC 0353 | MDR | 25 | 25 | 25 | 25 |
| 17 | CDC 0357 | MDR | 25 | 25 | 25 | 25 |
| 18 | CDC 0246 | MDR | 50 | 12.5 | 25 | 50 |
| 19 | CDC 0250 | MDR | 25 | 25 | 25 | 25 |
| 20 | CDC 0064 | MDR | 25 | 25 | 25 | 25 |
| 21 | ATCC 27853 | QC strain | 25 | 25 | 25 | 25 |

AB-101 Lots 01, 02 and a purified extract of taspine for MIC are compared. The concentration of taspine at the highest concentration tested (i.e. 50%, relative to AB-10) is 10 μg/mL demonstrated for the first time from a bacteriologic perspective, taspine does not have activity as evaluated by this invitro test method against MSSA and MRSA. Taspine may have additional synergistic benefits to be included in the whole extract in the final product for topical treatment of ABSSSI. Results are shown in Table 18.

TABLE 18

| | | | | | | MIC (% relative to | | | |
|---|---|---|---|---|---|---|---|---|---|
| | | MIC (% vol./vol.) | | | | amount in AB-101) | | MIC (μg/mL) | |
| Strain ID | Characteristic | AB-101 Lot 01 | | AB-101 Lot 02 | | Taspine | | Methicillin | Mupirocin |
| CDC 218 | MupirocinR, MRSA | 12.5 | 12.5 | 12.5 | 12.5 | >50 | >50 | >64 | >256 |
| CDC 224 | MupirocinR, MRSA | 12.5 | 12.5 | 12.5 | 12.5 | >50 | >50 | >64 | >256 |
| CDC 228 | MupirocinR, MRSA | 3.13 | 3.13 | 3.13 | 3.13 | >50 | >50 | 64 | >256 |
| 1674606 | MupirocinR, MSSA | 50 | 50 | 50 | 50 | >50 | >50 | 2-8 | >256 |
| 1674607 | MupirocinR, MRSA | 12.5 | 12.5 | 12.5 | 12.5 | >50 | >50 | >64 | >256 |
| 1674608 | MupirocinR, MSSA | 12.5 | 12.5 | 25 | 50 | >50 | >50 | 2-4 | >256 |
| 1674611 | MupirocinR, MRSA | 6.25 | 6.25 | 12.5 | 12.5 | >50 | >50 | 64 | >256 |
| CDC 480 | MupirocinS, MRSA | 12.5 | 25 | 12.5 | 25 | >50 | >50 | 32-64 | ≤0.25 |
| CDC 481 | MupirocinS, MRSA | 12.5 | 12.5 | 12.5 | 12.5 | >50 | >50 | 16-32 | ≤0.25 |
| CDC 482 | MupirocinS, MRSA | 12.5 | 12.5 | 12.5 | 12.5 | >50 | >50 | 8-32 | ≤0.25 |
| CDC 483 | MupirocinS, MRSA | 12.5 | 12.5 | 12.5 | 12.5 | >50 | >50 | 64 | ≤0.25 |
| CDC 220 | MupirocinS, MRSA | 12.5 | 12.5 | 6.25 | 12.5 | >50 | >50 | 64->64 | ≤0.25 |
| CDC 227 | MupirocinS, MRSA | 6.25 | 6.25 | 12.5 | 12.5 | >50 | >50 | 64 | ≤0.25-0.5 |
| CDC 461 | MupirocinS, MSSA | 12.5 | 12.5 | 12.5 | 12.5 | >50 | >50 | 4-8 | ≤0.25-1 |
| CDC 462 | MupirocinS, MSSA | 6.25 | 12.5 | 6.25 | 12.5 | >50 | >50 | 8 | ≤0.25 |
| CDC 463 | MupirocinS, MSSA | 6.25 | 6.25 | 12.5 | 12.5 | >50 | >50 | 8-16 | ≤0.25-0.5 |
| CDC 464 | MupirocinS, MSSA | 12.5 | 12.5 | 12.5 | 12.5 | >50 | >50 | 2 | ≤0.25 |
| CDC 484 | MupirocinS, MSSA | 12.5 | 12.5 | 12.5 | 12.5 | >50 | >50 | 2 | ≤0.25 |
| CDC 485 | MupirocinS, MSSA | 50 | 50 | 50 | 50 | >50 | >50 | 2-4 | ≤0.25 |
| ATCC 29213 | QC control | 12.5 | 50-12.5 | 12.5 | 12.5 | >50 | >50 | 1-2 | ≤0.25-0.5 |

AB-101 Lots 01, 02 and a purified extract of taspine for MBC are compared. Demonstrated for the first time from a bacteriologic perspective, taspine does not have activity as evaluated by this invitro test method against MSSA and MRSA. Taspine may have additional synergistic benefits to be included in the whole extract in the final product for topical treatment of ABSSSI. Results are shown in Table 19.

TABLE 19

| | | | | | | MBC (% relative to | |
|---|---|---|---|---|---|---|---|
| | | MBC (% vol./vol.) | | | | amount in AB-101) | |
| Strain ID | Characteristic | AB-101 Lot 01 | | AB-101 Lot 02 | | Taspine | |
| CDC 218 | MupirocinR, MRSA | 50 | 50 | 50 | 50 | >50 | >50 |
| CDC 224 | MupirocinR, MRSA | 50 | 50 | 50 | 50 | >50 | >50 |
| CDC 228 | MupirocinR, MRSA | 6.25 | 6.25 | 6.25 | 6.25 | >50 | >50 |
| 1674606 | MupirocinR, MSSA | >50 | >50 | >50 | >50 | >50 | >50 |
| 1674607 | MupirocinR, MRSA | 12.5 | 12.5 | 25 | 25 | >50 | >50 |
| 1674608 | MupirocinR, MSSA | 50 | 50 | 50 | >50 | >50 | >50 |
| 1674611 | MupirocinR, MRSA | 12.5 | 12.5 | 50 | 50 | >50 | >50 |
| CDC 480 | MupirocinS, MRSA | 50 | 50 | 50 | 50 | >50 | >50 |
| CDC 481 | MupirocinS, MRSA | 50 | 50 | 50 | 50 | >50 | >50 |
| CDC 482 | MupirocinS, MRSA | 50 | 50 | 50 | 50 | >50 | >50 |
| CDC 483 | MupirocinS, MRSA | 50 | 50 | 50 | 50 | >50 | >50 |
| CDC 220 | MupirocinS, MRSA | 25 | 25 | 25 | 25 | >50 | >50 |
| CDC 227 | MupirocinS, MRSA | 12.5 | 12.5 | 25 | 25 | >50 | >50 |
| CDC 461 | MupirocinS, MSSA | 50 | 50 | 50 | 50 | >50 | >50 |
| CDC 462 | MupirocinS, MSSA | 12.5 | 50 | 50 | 50 | >50 | >50 |
| CDC 463 | MupirocinS, MSSA | 12.5 | 12.5 | 12.5 | 12.5 | >50 | >50 |
| CDC 464 | MupirocinS, MSSA | >50 | 50 | 50 | 50 | >50 | >50 |
| CDC 484 | MupirocinS, MSSA | 50 | 50 | 50 | 50 | >50 | >50 |
| CDC 485 | MupirocinS, MSSA | 50 | 50 | 50 | 50 | >50 | >50 |
| ATCC 29213 | QC control | 50-12.5 | 50-12.5 | 50 | 50 | >50 | >50 |

### Example 4—Log P and In Vitro Permeation Test (IVPT) Characterization of AB-101

A Log P analysis and in vitro Permeation Test IVPT tests were conducted to assess AB-101's effectiveness in the bronchial passageway. Log P characterizes the partitioning coefficient of AB-101 and the IVPT is a critical tool for understanding drug delivery into the various layers of skin, inclusive of epithelial skin and can aid in formulation selection. All reference to skin includes references to epithelial skin and epithelial layer of the lungs, nose, mouth and throat. IVPT is a flux measurement that is common to those skilled in the art to evaluate topical drugs inclusive to topical delivery to the lungs, nose, mouth and throat.

Log P is an assessment of a molecule's lipophilicity, indicating how readily an analyte can partition between aqueous and organic phases. When a topical drug possesses poor lipophilicity, with Log P rating of −1 to 2 (Table 20), when it is applied to the skin the topical drug will not penetrate or disperse easily into the skin. To reach high systemic levels, a topical drug must be permeable enough, which is defined as lipophilic enough, to partition into the lipid bilayer of the skin, but, not so lipophilic that once it is in the skin bilayer it will stay sequestered there. Lipophilicity plays a major role in determining where drugs are distributed within the body after absorption into tissues and, as a consequence, in how rapidly they are metabolized and excreted.

TABLE 20
LogP Rating Scale and Values
LogP:

| | | | | | | | |
|---|---|---|---|---|---|---|---|
| −1.0 | 0.0 | 1.0 | 2.0 | 3.0 | 4.0 | 5.0 | 6.0 |

| | | |
|---|---|---|
| Limited permeability, poor | Intermediate | Highly permeable, |
| lipid solubility, poor skin | permeability, able to | crosses into skin and |
| absorption and distribution. | cross the skin lipid | stays sequestered in |
| | bilayer but not be | skin |
| | sequestered in the | |
| | skin | |

The Log P value is a constant defined in the following manner: Log P=log 10 (Partition Coefficient) Partition Coefficient, P=[organic]/[aqueous] Where [ ] indicates the concentration of solute in the organic and aqueous partition. A negative value for log P means the compound has a higher affinity for the aqueous phase (it is more hydrophilic); when log P=0 the compound is equally partitioned between the lipid and aqueous phases; a positive value for log P denotes a higher concentration in the lipid phase (i.e., the compound is more lipophilic). Log P=1 means there is a 10:1 partitioning in Organic: Aqueous phases. Although log P is a constant, its value is dependent on the choice of the organic partitioning solvent and, to a lesser degree, on the conditions of measurement. AB-101 partition coefficient was determined using water and n-Octanol.

The partitioning assays reported here (Table 30) determined the in vitro partitioning and release of AB-101's five selected components (the four PAC's catechin, epicatechin, epigallocatechin, gallocatechin, plus taspine) from four different AB-101 forms: (1) AB-101 100% from AB-101 batches 0, 1, 2 and X, (2) AB-101 100% from batches 0, 1, 2 and X after they were lyophilized and resuspended in water to determine the impact on partitioning of the lyophilization and resuspension processes in the event these processes are utilized in commercial production. These data report poor lipophilicity of all five selected AB-101 components (the PAC's and taspine) as defined by Log P analysis for all four forms tested. As the skin is lipophilic, the AB-101 PAC's and taspine and the other compounds of AB-101 being poorly lipophilic will not penetrate or disperse easily into the skin when applied directly to the skin. Lack of lipophilicity further demonstrates the AB-101 PAC's and taspine and the other compounds of AB-101 will not be passed through the skin into the underlying tissues or systemic circulation. The AB-101 PAC's and taspine AB-101 poor skin and tissue penetration demonstrates their limited ability to be absorbed into the skin resulting in even lower likelihood they will pass through the skin to reach systemic circulation or other tissued or organs. If any of the AB-101 PAC's or taspine do reach systemic circulation, their poor tissue penetration means they will not be accumulated in tissues and will be excreted from the body. Results are shown in table 21.

TABLE 21
LogP Values for specified components components of AB-
101 in Four Forms: AB-101 100%, AB-101 100% Resuspended

| | | LogP | LogP | LogP | LogP | LogP |
|---|---|---|---|---|---|---|
| AB-101 | AB-101 | Catechin¹ | Epicatechin¹ | Gallocatechin¹ | Epigallocatcchin¹ | Taspin¹ |
| Form | Batch # | (0.51-1.8) ² | (0.4-1.8) ² | (0.71-1.41) ² | (0.00-1.49) ² | (0.99-2.49) ² |
| AB-101, 100% | Batch 0 | 0.27 | −0.09 | −0.24 | −0.79 | −1.15 |
| Lyophilized & | | 0.39 | 0.02 | 0.36 | 1.20 | −1.06 |
| Resuspended | | | | | | |
| AB-101, 100% | Batch 1 | 0.29 | −0.02 | 0.01 | 0.27 | −1.76 |
| Lyophilized & | | 0.23 | 0.02 | −0.13 | −0.11 | −0.94 |
| Resuspended | | | | | | |
| AB-101, 100% | Batch 2 | 0.59 | 0.13 | 0.56 | 0.86 | −0.90 |
| Lyophilized & | | 0.07 | −0.17 | −0.48 | −1.71 | −1.71 |
| Resuspended | | | | | | |
| AB-101, 100% | Batch X | −0.14 | 0.07 | 0.03 | 1.82 | −0.31 |
| Lyophilized & | | −0.13 | 0.09 | −025 | −0.55 | −0.07 |
| Resuspended | | | | | | |

¹LogP values for reference standard PACs and Taspine do not significantly differ from PACs and Taspine from AB-101 tested in multiple forms
² Reference standard LogP values from ALOGPS, ChemAxon, Chemspider

In vitro Permeation Test, IVPT, with AB-101 were designed to assess the in vitro partitioning and transdermal release of catechin, epicatechin, gallocatechin, epigallocatechin, and taspine from AB-101 100% based on the in vitro release testing. In vitro testing such as this is an accepted, industry-standard method for determining the skin penetration of a topically applied products (USP Chapter 1724, USP 36/NF31, 2013), and in vitro testing using Strat-M membranes has been shown to be predictive of in vivo results (Flaten et al, 2005). The Franz diffusion cell model is a well establish industry and academic method for performing permeation testing (Ueda et al, 2009; Li & Rahn, 1993). To determine the flux (ug/cm²/hr) of catechin, epicatechin, gallocatechin, epigallocatechin, and taspine into and through the epithelial skin, an existing analytical LC-MS/MS method was used.

IVPT data, shown in Table 22, revealed poor transdermal permeation of these compounds from AB-101 100%. As defined by flux, AB-101 lots (X and 1) demonstrated low amounts permeating across all 5 compounds tested.

TABLE 22
Flux Calculations

| Vehicle | C | EC | GC | EGC | T |
|---|---|---|---|---|---|
| Lot 1 | 8.82 | 9.11 | 18.25 | 503.51 | 10.01 |
| Lot X | 1.53 | 1.04 | 4.33 | 57.09 | 16.69 |

Flux (J): ug/cm²/hr.
Flux is the amount of permeant crossing a membrane per unit time
J = Q/(A [symbol] t) [symbol]
compound [symbol]
A = area of membrane
T = time [symbol]
indicates data missing or illegible when filed

Taken together, the Log P and IVPT results indicate that the 4 proanthocyanidins and taspine demonstrate low accumulation, flux and permeability across intact synthetic membranes. Any permeation that does occur would be unlikely to distribute systemically within the body at significant levels due to the poor distribution properties revealed from the in vitro Log P analysis. The results of these studies support the claim that the 4 PACs and taspine measured are unlikely to reach toxic amounts at a systemic level based on these in vitro results.

### Example 5—Polydispersity Index Analysis of AB-101

To determine the PDI, an HPLC-UV method using gel permeation chromatography (GPC) and/or size exclusion (SEC) chromatography for this polymer analysis was developed. The polymer size distribution is calculated using the following equation: PDI=M_{w}/Mₙ, where M_{w} is weight average molecular weight and Mₙ is the number of the average molecular weight.

Sample Preparation: AB-101 was prepared by aliquoting 5 mL of material and lyophilizing the samples overnight. Samples were then weighed out and re-suspended in water at a concentration of 5 mg/mL. Samples were then diluted in water prior to GPC-UV/CAD analysis for PDI determination. Details of the HPLC method are below:
Column: Jordi Resolve columns 5 μm (7.8 mm ID×300 mm L), Porosity: 500 Å
HPLC Conditions: Isocratic: Dimethylformamide (DMF)
Flow rate: 0.7 mL/min
Injection volume: 20 μL
Column temperature: 40° C.
Detection: CAD
Standards: Polymethyl methacrylate (PMMA) EasiVials (Agilent)

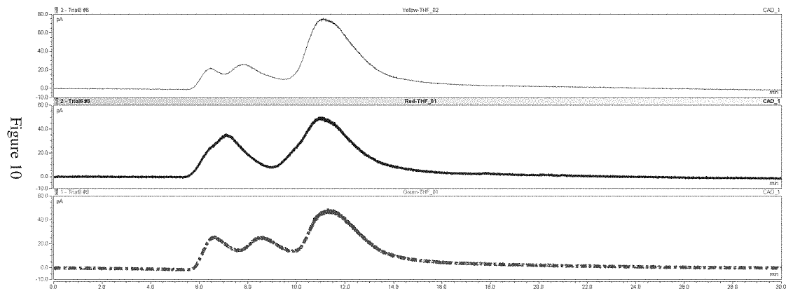

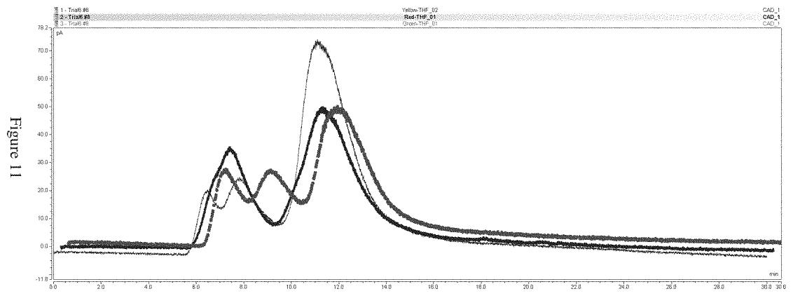

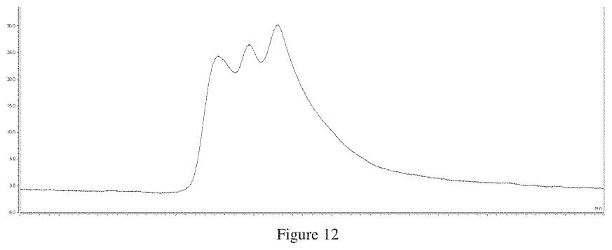

FIGS. 10 and 11 show the gel permeation chromatogram of 3 PMMA standards analyzed. Top line is standard Yellow. Thicker line in the middle is standard Red. Dashed line on the bottom is standard Green. These standards were run at 2 mg/mL. The chromatograph demonstrated discerning peaks that enables calculation of PDI. FIG. 12 depicts the AB-101 Lot 01 chromatograms at a 1.25 mg/mL concentration.

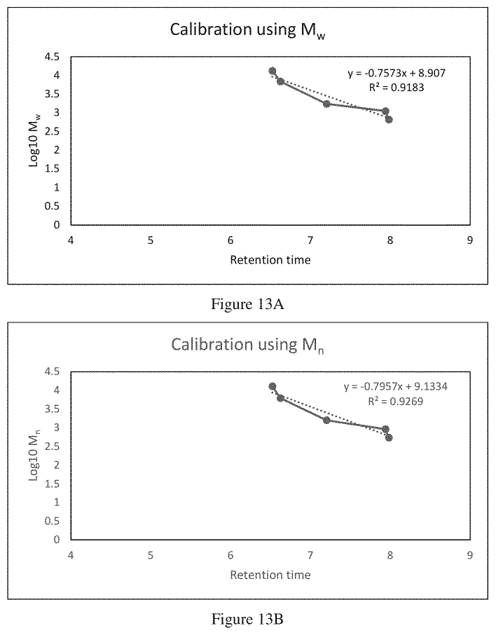

Polydispersity Index Calculations: Sample analysis for AB-101 Lot 01 using the developed method was performed. Calibration curves were generated based on Log M_{w} vs. retention time and Log Mₙ vs. retention time. The calibration curves are shown in FIGS. 13A and 13B. Using the calibration curves, the PDI was able to calculate the M_{w} and Mₙ values from AB-101 Lot 01 based on the peak retention times. Results are shown in Table 23.

TABLE 23

| RT (min) | Log M_{w} | M_{w} | Log Mₙ | Mₙ |
|---|---|---|---|---|
| 6.8 | 2.78 | 1.02 | 2.93 | 1.08 |
| 7.8 | 1.46 | 0.38 | 1.68 | 0.52 |
| 8.0 | 1.20 | 0.18 | 1.42 | 0.35 |
| | Average | 0.53 | Average | 0.65 |
| | | | PDI | 0.81 |

Lot 01 of AB-101 has a PDI of 0.81. This is in contrast to the PDI range of 0.9-1.2 for crofelemer as disclosed in WO 2012/101008. The larger PDI for crofelemer indicates that through the refining and fractionation process, crofelemer has greater heterogeniety in cross linking, network formation, chain length and branching than AB-101.

1. A method of treating a cystic fibrosis and/or a bacterial infection in a cystic fibrosis patient in a subject in need thereof comprising administering to the lungs via inhalation a therapeutically effective amount of a pharmaceutical composition containing filtered latex of Croton lechleri, wherein the Croton lechleri contains at least about 110 PPM of Gallocatechin, at least about 780 PPM of Epigallocatechin, about at least about 1.6 PPM of Catechin at least about 2 PPM of Epicatechin, at least about 45 PPM Taspine, and at least about 0.1 PPM of Dimethylcedrusin, wherein the therapeutically effective amount of filtered latex of Croton lechleri Müll.Arg is about 3 wt % to 90 wt %, and the Croton lechleri has a polydispersity index of about 0.5 to 0.85, wherein the composition is suitable for administration via inhalation.

2. The method of claim 1, wherein the Croton lechleri is Croton lechleri Müll.Arg.

3. The method of claim 1, wherein the bacterial infection is selected from the group consisting of a Pseudomonas aeruginosa infection, aMDR resistant Pseudomonas aeruginosa infection, a Staphylococcus aureus infection, a methicillin-resistant Staphylococcus aureus infection, and combinations thereof.

4. The method of claim 3, wherein the bacterial infection is a Pseudomonas aeruginosa infection.

5. The method of claim 3, wherein the bacterial infection is a MDR resistant Pseudomonas aeruginosa infection.

6. The method of claim 3, wherein the bacterial infection is a Staphylococcus aureus infection.

7. The method of claim 3, wherein the bacterial infection is a methicillin-resistant Staphylococcus aureus infection.

8. The method of claim 1, wherein the pharmaceutical composition is a liquid.

9. The method of claim 1, wherein the pharmaceutical composition is a powder.

10. The method of claim 1 wherein the administration is until the bacterial infection in a cystic fibrosis patient is treated.

11. The method of claim 1, wherein the pharmaceutical composition is administered to the lungs via oral inhalation.

12. The method of claim 1, wherein the pharmaceutical composition is administered to the lungs via nasal inhalation.

13. The method of claim 1, wherein the pharmaceutical composition further comprises a pharmaceutically acceptable carrier.

14. The method of claim 1, wherein the filtered latex of Croton lechleri has a PDI of about 0.81.

15. A pharmaceutical composition comprising a therapeutically effective amount of filtered latex of Croton lechleri Müll.Arg, wherein the Croton lechleri Müll.Arg contains at least about 110 PPM of Gallocatechin, at least about 780 PPM of Epigallocatechin, at least about 1.6 PPM of Catechin, at least about 2 PPM of Epicatechin, at least about 45 PPM Taspine, at least about 0.1 PPM of Dimethylcedrusin and wherein the pharmaceutical composition is suitable for administration to the lungs.

16. The pharmaceutical composition of claim 15, wherein the pharmaceutical composition is a liquid.

17. The pharmaceutical composition of claim 15, wherein the pharmaceutical composition is a powder.

18. The pharmaceutical composition of claim 15, wherein the therapeutically effective amount of filtered latex of Croton lechleri Müll.Arg is about 3 to 90 wt %.

19. The pharmaceutical composition of claim 15, wherein the pharmaceutical composition further comprises a pharmaceutically acceptable carrier.

20. The pharmaceutical composition of claim 15, wherein the pharmaceutical composition has a MIC of at least 50% concentration of AB-101.

21. The pharmaceutical composition of claim 15, wherein the filtered latex of Croton lechleri has a PDI of about 0.81.
